(19) United States
(12) Patent Application Publication
(10) Pub. No.: US 2023/0041859 A1
(43) Pub. Date: Feb. 9, 2023

(54) USE OF TERPENOIDS IN THE TREATMENT OR PREVENTION OF FIBROTIC DISEASES

(71) Applicant: Arjil Biotech Holding Company Limited, Hsinchu City (TW)

(72) Inventors: Yeh-B WU, Hsinchu City (TW); Jir-Mehng LO, Hsinchu City (TW); Hui-Ju LIANG, Hsinchu City (TW); Pei-Hsin LIN, Hsinchu City (TW); Kuo-Kuei HUANG, Hsinchu City (TW)

(21) Appl. No.: 17/365,602

(22) Filed: Jul. 1, 2021

(51) Int. Cl. A61K 31/575; A61K 36/06; A61P 13/12; A61P 1/16; A61P 11/00

(52) U.S. Cl. CPC A61K 31/575; A61K 36/06; A61P 13/12; A61P 1/16; A61P 11/00; A61K 2236/33

(57) ABSTRACT

The present invention relates to a method of preventing or treating a fibrotic condition, comprising administering an effective amount of composition to a subject in need thereof; wherein the composition comprises triterpenes extracted from Antrodia camphorate or Anisomeles indica.

### FIELD OF THE INVENTION

The invention relates to herbal terpenoids from Antrodia camphorata and anisomeles indica extract, particularly to a medicinal and edible formula to moderate fibrotic diseases effectively.

### BACKGROUND OF THE INVENTION

Fibroproliferative disorders are troubling problems for an increasing number of individuals and is a common pathological sequela of many persistent inflammatory diseases, such as pulmonary fibrosis, progressive kidney disease, liver cirrhosis, atherosclerosis and benign prostatic hyperplasia.

Impaired renal repair after acute kidney injury induces fibrosis which may ultimately lead to the development of chronic kidney disease. Kidney injury activates multipotent progenitor cells to repair tissue. However, these cells become dysfunctional and induce fibrogenic repair as the injury sustains initiating kidney fibrosis. The pathogenesis of renal fibrosis is a progressive process that ultimately leads to end-stage renal failure, a devastating disorder that requires dialysis or kidney transplantation.

Non-alcoholic fatty liver disease (NAFLD) is a leading form of chronic liver disease with large unmet need. Non-alcoholic steatohepatitis (NASH), a progressive variant of NAFLD, can lead to fibrosis, cirrhosis, and hepatocellular carcinoma. NAFLD and NASH are entities that are becoming subject of interest of the medical community in general, especially because of the increased prevalence of diabetes and obesity in the world population. Clinical evaluation of every patient with abnormal aminotransferase levels should take into account non-alcoholic fatty liver and its spectrum, especially if the subject is obese or diabetic. The prognosis of simple NAFLD is generally benign, but if there is fibrosis, ballooning of the hepatocytes, inflammation and Mallory bodies there is risk to progression to cirrhosis.

Autoimmune hepatitis (AIH) is a chronic liver disease without a clear etiology but can be characterized by hepatocellular inflammation. Severe AIH may progress into liver cirrhosis, hepatocellular carcinoma, or even death. Cirrhosis develops in as many as 40% of treated patients with autoimmune hepatitis depending on the length of observation. Anti-fibrotic therapies are emerging that can supplement the anti-inflammatory and immunosuppressive actions of current regimens, and these regimens promise to re-direct the objectives of treatment in autoimmune hepatitis to the prevention, stabilisation and reversal of hepatic fibrosis.

Atherosclerosis, which is one of the primary causes of the development of cardiovascular disease, is associated with vascular fibrosis. Vascular fibrosis involves accumulation of extracellular matrix (ECM) proteins, particularly collagen and fibronectin in the vascular media and contributes to structural remodeling and scar formation. A lack of elastin or excessive collagen in the vascular wall leads to vascular fibrosis and increased stiffness.

In benign prostatic hyperplasia, the deposition of collagen fibers in prostate is for replacing broken myofibers, however results in stiffness and weakness of the muscular tissue and deposition of prostatic fluid in gland tubes. Prostatic fibrosis plays a central role in the development of bladder outlet obstruction in aging men.

Medicinal fungus Antrodia camphorata (AC) is a well-known Chinese folk medicine, known to possess numerous biological activities, especially an anti-tumor effect in in vitro cancer cells and in vivo animal models. It is considered an efficient alternative phyto-therapeutic agent or an adjuvant to cancer treatment and immune-related diseases given its diverse bioactive compounds. To date, a total of 225 compounds have been isolated, identified, and structurally elucidated, including macromolecules (nucleic acids, proteins, and polysaccharides), small molecules (benzenoids, lignans, benzoquinones, and maleic/succinic acid derivatives), terpenoids (lanostane triterpenes, ergostane triterpenes, diterpenes, monoterpenes, and steroids), nucleotides (nucleobase and nucleoside), fatty acids, and fatty acid esters.

Cumulative in vitro and in vivo studies have revealed its anti-diabetic and anti-hyperlipidemic, anti-hypertensive, anti-inflammatory, antioxidant, antimicrobial, cardiovascular disease preventive, immunomodulatory, hepatoprotective, and neuroprotective effects. However, the efficacy of Antrodia camphorata and its components in the treatment of fibrosis has not been evaluated.

Anisomeles indica commonly known as ‘Indian Catmint’ is a source of medicinally active compounds and have various pharmacological effects. The plant is used traditionally as an analgesic, anti-inflammatory and in skin problems. Medicinally it has been proven to possess various pharmacological activities like antioxidant, antimicrobial, anti-HIV, anti-Helicobacter pylori and anti-cancer activity. It is also used in chronic rheumatism. Further studies reveal the presence of various phytochemical constituents mainly triterpenes, β-sitosterol, stigmasterol, flavones, apigenin and ovatodiolides etc.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts the separation of antcin K, dehydrosulphurenic acid/sulphurenic acid, versisponic acid D and dehydroeburicoic acid from Antrodia camphorata extract

FIGS. 2A-2C Protective effects of Antrodia camphorata Extract and Compounds on cisplatin-induced kidney damage in AKI mice. To analyze the effects of Antrodia camphorata Extract and compounds, mice are given daily for 7 days, starting from 3 weeks after first dose of cisplatin, and sacrificed at 4 weeks. The morphological changes in the kidneys (A). Blood urea nitrogen (BUN) levels (B). Serum creatinine (CRE) levels (C). The data are presented as the means±S.E.M (n=5). ### denotes p<0.001 compared with sample of control group. ** p<0.01 and *** p<0.001 compared with cisplatin group.

FIG. 3 Protective effects of Antrodia camphorata Extract and Compounds on cisplatin-induced kidney damage in AKI mice. To analyze the effects of Antrodia camphorata Extract and compounds, mice are given daily for 7 days, starting from 3 weeks after first dose of cisplatin, and sacrificed at 4 weeks. Kidneys stained with H&E. After cisplatin challenge, kidneys in each group are prepared for histological evaluation. Representative histological section of the kidneys is stained by H&E staining, magnification (400×). The data are presented as the means±S.E.M (n=5). ### denotes p<0.001 compared with sample of control group. ** p<0.01 and *** p<0.001 compared with cisplatin group. Tubular cell necrosis is marked with arrows; the bar indicates 50 μm.

FIGS. 4A-4E Antrodia camphorata Extract and Compounds regulated (A) TNF-α, (B) IL-1β, (C) IL-6 (D) TGF-β and (E) albumin in serum. Serum levels of TNF-α, IL-1β, IL-6, TGF-β and albumin are determined by commercial ELISA kits. Data are represented as mean±S.E.M. (n=5). ### denotes p<0.001 compared with sample of control group. ** p<0.01 and *** p<0.001 compared with cisplatin-only group.

FIGS. 5A-5B Effects of ARH005-EA (A) and ARH (B) on cisplatin-induced TWEAK, α-SMA, P53 and P21 signaling expression in kidneys. Protein levels of TWEAK, α-SMA, P53 and P21 protein expression in kidney homogenates are evaluated by western blot analysis after cisplatin challenge.

FIG. 6 depicts the process of CCl₄-induced fibrosis model

FIGS. 7A-7C depict (A)delta weight, (B)liver weight, and (C)liver/body-weight ratio, respectively.

FIGS. 8A-8C depict serum levels of (A)AST, (B)ALT, and (C)AST/ALT respectively in rats following CCl₄-induced hepatic damage.

FIGS. 9A-9E depict (A)inflammation, (B)vacuolation, (C)necrosis, (D)fibrosis, (E)total histological score in liver.

FIG. 10 Representative histological section of the liver is stained by H&E staining.

FIG. 11 depicts the process of Con A (concanavalin A) induced acute hepatitis model.

FIGS. 12A-12C depict the process of Ovatodiolide (AR100-DS1) on GOT, GPT and body weight. (A) Serum GOT and (B) serum GPT 24 h after 15 mg/kg Con A challenge. (C) body weight before and after 15 mg/kg Con A challenge. Data are presented as mean±SEM (n=9). * p<0.05 versus Veh by the t test. Veh, vehicle; Dex, dexamethasone.

FIGS. 13 Effects of Ovatodiolide (AR100-DS1) on liver injury. Liver histopathology of (A) Naïve, (B) 15 mg/kg Con A (Veh), (C) 2019-0321-1 and (D) dexamethasone, and (E) histopathological scores of necrosis. Data are presented as mean±SEM (n=9). *** p<0.001 versus Veh by the Student's t test. Veh, vehicle; Dex, dexamethasone.

FIG. 14 depicts the process of atherosclerosis rabbit model.

FIG. 15 depicts initial and final average body weight of rabbits. † and * indicate a P<0.05 as compared with the control group and HF group, respectively.

FIGS. 16A-16C depict changes in AST, ALT, BUN between WO groups in each group of rabbits † and * indicate a P<0.05 as compared with the control group and HF group, respectively.

FIGS. 17A-17D depict changes in TG, TC, HDL-C, LDL-C between WO groups in each group of rabbits † and * indicate a P<0.05 as compared with the control group and HF group, respectively.

FIGS. 18A-18C depict changes in AST, ALT, BUN between W4 groups in each group of rabbits † and * indicate a P<0.05 as compared with the control group and HF group, respectively.

FIGS. 19A-19D depict changes in TG, TC, HDL-C, LDL-C between W4 groups in each group of rabbits. † and * indicate a P<0.05 as compared with the control group and HF group, respectively.

FIGS. 20A-20C depict changes in AST, ALT, BUN between W8 groups in each group of rabbits † and * indicate a P<0.05 as compared with the control group and HF group, respectively.

FIGS. 21A-21D depict changes in TG, TC, HDL-C, LDL-C between W8 groups in each group of rabbits. † and * indicate a P<0.05 as compared with the control group and HF group, respectively.

FIGS. 22A-22C depict changes in AST, ALT, BUN between W12 groups in each group of rabbits † and * indicate a P<0.05 as compared with the control group and HF group, respectively.

FIGS. 23A-23D depict changes in TG, TC, HDL-C, LDL-C between W12 groups in each group of rabbits. † and * indicate a P<0.05 as compared with the control group and HF group, respectively.

FIG. 24 depict histopathochemical examination of aortic fatty streak lesions in the hypercholesterolemic rabbit model after the 12-week study.

FIG. 25 depicts HE staining of coronary artery sections after sacrifice in each group of rabbits.

FIG. 26 depicts HE staining of coronary arteries after sacrifice in each group of rabbits. N, neointima layer; M, media layer.

FIG. 27 depicts the manifestation of vascular restenosis is presented as the ratio of neointima-to-media area (N/M ratio) N, neointima layer; M, media layer. *p<0.05, ** p<0.01, and *** p<0.001 compared with HFD group, respectively.

FIG. 28 depicts histopathochemical examination of heart tissues in the hypercholesterolemic rabbit model after the 12-week study.

FIG. 29 depicts photographs of liver appearance in the hypercholesterolemic rabbit model after the 12-week study.

FIG. 30 depicts histopathochemical examination of liver tissues in the hypercholesterolemic rabbit model after the 12-week study.

FIG. 31 depicts the animal's body weight and lung weight.

FIG. 32 depicts histopathological changes of the lungs in bleomycin-induced lung fibrosis in mice.

FIG. 33 depicts Masson's Trichrome staining of the lung in bleomycin-induced lung fibrosis in mice.

FIG. 34 depicts the effects of A. camphorata Extract and Compounds on hydroxyproline content in pulmonary damage of mice induced by bleomycin.

FIGS. 35A-35D depict A. camphorata Extract and compounds regulated (A) TNF-α, (B) (C) IL-6 (D) and TGF-β(E) in BALF.

FIG. 36 depicts the effects of A. camphorata Extract and compounds regulated on lung MPO activity in BLM-induced mice.

FIG. 37 HepG2 cells were treated with different concentrations of oleic acid (OA) to induce steatosis.

FIG. 38A-38B depicts 24 h-OA-induced steatosis in HepG2 cells.

FIG. 39 depicts cytotoxicity test of compounds (3.7, 11.1, 33.3, 100 and 200 μg/mL) in HepG2 cells.

FIG. 40 depicts Anti-steatosis test of compounds in HepG2 cells.

### DETAILED DESCRIPTION OF THE INVENTION

For the convenience of the description of the present invention, the central idea expressed in the above summary of the invention is expressed by way of specific examples. Various items in the embodiments are depicted in terms of ratios, dimensions, amounts of deformation, or displacements that are suitable for illustration, and are not drawn to the proportions of actual elements, as set forth above.

The term “terpenes” refers to a large and diverse class of organic compounds, whose basic structure follows a general principle: 2-Methylbutane residues, less precisely but usually also referred to as isoprene units, (C₅)ₙ, build up the carbon skeleton of terpenes. About 30 000 terpenes are known at present in the literature. Depending on the number of 2-methylbutane (isoprene) subunits one differentiates between hemi- (C₅), mono- (C₁₀), sesqui- (C₁₅), di- (C₂₀), sester- (C₂₅), tri- (C₃₀), and tetraterpenes (C₄₀).

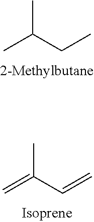

The terms “subject,” “individual.” “host, and “patient” are used interchangeably herein to refer to a living animal, including a human and a non-human animal. The Subject may, for example, be an organism possessing immune cells capable of responding to antigenic stimulation, and stimulatory and inhibitory signal transduction through cell Surface receptor binding. The Subject may be a mammal. Such as a human or non-human mammal, for example, dogs, cats, pigs, cows, sheep, goats, horses, rats, and mice. The term ‘subject does not preclude individuals that are entirely normal with respect to a disease, or normal in all respects.

The term “treatment” refers to a therapeutic or preventative measure. The treatment may be administered to a subject having a medical disorder or who ultimately may acquire the disorder, in order to prevent, cure, delay, reduce the severity of or ameliorate one or more symptoms of a disorder or recurring disorder, or in order to prolong the Survival of a subject beyond that expected in the absence of such treatment.

The term “therapeutically effective amount’ means the amount of the subject compound that may elicit a desired response, for example, a biological or medical response of a tissue, System, animal, or human that is sought, for example, by a researcher, veterinarian, medical doctor, or other clinician.

Determination of biochemical parameters Serum creatinine and serum urea are assessed using colorimetric kits according to manufacturer's instructions. Kits of the former markers are purchased from (HUMAN Diagnostics Worldwide, Magdeburg, Germany) with a chemical analyzer (Roche Diagnostics, Cobas Mira Plus, Rotkreuz, Switzerland).

Kidney histopathology The anterior portion of the left lateral liver lobe from each mouse is fixed in 10% formaldehyde phosphate buffer, embedded in paraffin, cut into 5 μm sections, and then treated with an hematoxylin and eosin (H&E) stain for histological examination under the light microscopy (Nikon, ECLIPSE, TS100, Tokyo, Japan). Images are captured with a digital camera (NIS-Elements D 2.30, SP4, Build 387) at an original magnification of 400×.

TNF-α, IL-6, and IL-1β cytokines in serum The serum concentration of the pro-inflammatory cytokines (i.e. tumor necrosis factor-α (TNF-α), interleukin-6 (IL-6), and IL-1β) in serum are assessed with relevant enzyme-linked immunosorbent assay (ELISA) kits (Biosource International Inc., Sunnyvale, Calif., USA), based on the manufacturer's instructions.

Western blot analysis of the kidney tissues Lysis buffer, composed of 0.6% NP-40, 150 mM NaCl, 10 mM HEPES (pH 7.9), 1 mM EDTA, and 0.5 mM PMSF, is used in the homogenization of liver tissues at 4° C. The homogenized samples are then centrifuged at 3000 revolutions per minute (rpm) at 4° C. for 10 min to obtain the supernatant. The equal total cellular protein amounts of supernatant are determined by the protein standard of bovine serum albumin (BSA). Protein samples (50 μg) are resolved by denaturing 10% sodium dodecyl sulfate-polyacrylamide gel electrophoresis (SDS-PAGE) using standard methods, and then are transferred onto PVDF membranes (Immobilon, Millipore, Bedford, Mass., USA) for electroblotting and blocking with 10% skim milk. The membranes are incubated with an appropriate dilution of specific primary antibodies at 4° C., washed three times with TBS/tween (TBST) buffer, and subsequently incubated for 1 h at 37° C. with horseradish peroxidase-conjugated secondary antibodies (overnight). The membranes are washed three times before examination for immuno-reactive proteins by enhanced chemiluminescence (ECL) reagent (Thermo Scientific, Hudson, N.H., USA). Band intensity on scanned films are quantified and represented as relative intensity by comparing with the control group using Image J Software (NIH, Bethesda, Md., USA).

Statistical analysis Data obtained from animal experiments are expressed as the means and standard errors of the means (±S.E.M.). Student's t-test are used to examine the differences among multiple groups or between two groups. Statistical significance is expressed as * p<0.05, ** p<0.01 and *** p<0.001.

### Example 1. Preparation of Antrodia camphorata Extract

100 grams of Antrodia camphorata fruiting body is reflux with methanol for 6 hours, and the extract is collected and dried, thereby obtaining a total of 15 grams of methanol extract of Antrodia camphorata.

### Example 2. Preparation of Active Ingredients: Antcin K, Dehydrosulphurenic Acid/Sulphurenic Acid, Versisponic Acid D and Dehydroeburicoic Acid

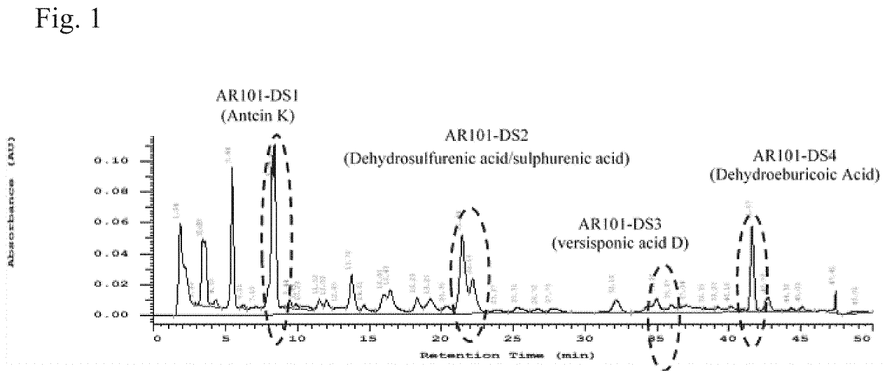

The methanol extract of Antrodia camphorata is further separated by silica column chromatography using n-hexane/ethyl acetate/methanol as eluent to provide the fractions (shown in FIG. 1):

### ARH101-DS1 (RS-Antcin K),

ARH101-DS2 (Dehydrosulphurenic acid/Sulphurenic acid),
ARH101-DS3 (Versisponic acid D) and

### ARH101-DS4 (Dehydroeburicoic Acid).

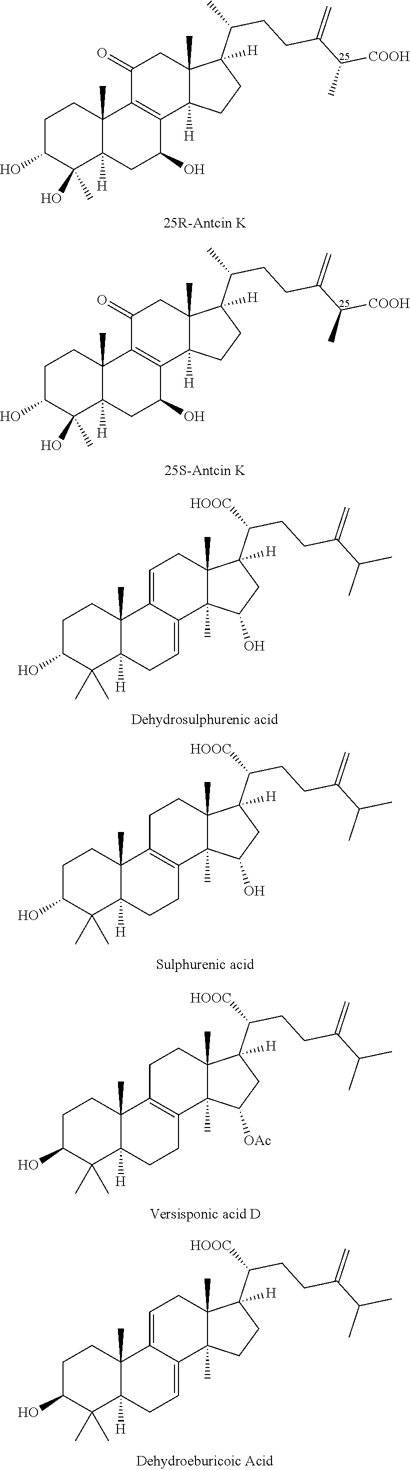

### Example 3. Preparation of AR003 Extract

100 grams of Antrodia camphorata (petri dish culture) is reflux with methanol for 6 hours, and the extract is collected and dried under reduced pressure to obtain 15 grams of the Antrodia camphorate ARH003 extract.

### Example 4. Preparation of AR003-E Extract

200 grams of Antrodia camphorata (petri dish culture) is reflux with ethanol for 6 hours, and the extract is collected and dried, thereby obtaining a total of 18 grams of ethanol extract of AR003-E Antrodia camphorata.

### Example 5. Preparation of AR004 Extract

100 grams of Antrodia camphorata (wood culture) is reflux with methanol for 6 hours, and the extract is collected and dried under reduced pressure to obtain the Antrodia camphorate ARH004 extract.

### Example 6. Preparation of AR005-EA Extract

100 grams of Antrodia camphorata (solid culture) is reflux with ethyl acetate for 6 hours, and the extract is collected and dried, thereby obtaining a total of 12 grams of EA extract of Antrodia camphorata.

### Example 7. Preparation of Anisomeles Indica Extract

The anisomeles indica extract is prepared by the following process: (1) An ethanol extract of anisomeles indica is taken, added into a silica-filled chromatographic column, and subjected to a gradient elution with the eluents “n-hexane/ethyl acetate”, “hexane/ethyl acetate/methanol” and “methanol” to obtain an anisomeles indica parting liquid. (2) The anisomeles indica parting liquid is separated by using silica-filled chromatographic column, and subjected to a gradient elution with the eluents “dichloromethane”, “dichloromethane/methanol” and “methanol” to obtain a separated concentrating substance; (3) the separated concentrating substance is recrystallized with the solvent “n-hexane/ethyl acetate” to obtain the anisomeles indica crystallite.

### Example 8. Preparation of Active Ingredients: Ovatodiolide (AR100-DS1)

200_{g} of ethanol extract of anisomeles indica is taken, added into a silica-filled chromatographic column (10×15 cm), and subjected to a gradient elution with 1200 ml of each of the eluents: “n-hexane/ethyl acetate (with a ratio of 10/1, 5/1, 3/1, 1/1)”, “hexane/ethyl acetate/methanol (with a ratio of 6/4/1, 3/2/1)” and “methanol” to obtain 140 g of an initial parting liquid.

The 140_{g} of the initial parting liquid is separated by using silica-filled chromatographic column (10×15 cm), and subjected to a gradient elution with 1000 ml of each of the eluents: “dichloromethane”, “dichloromethane/methanol (with a ratio of 10/1, 5/1, 7/3)” and “methanol” to obtain a separated concentrating substance. The separated concentrating substance is further recrystallized with the solvent: “n-hexane/ethyl acetate” to obtain a crystal. The crystal is identified as a diterpenoid compound whose chemical structure is ovatodiolide by a nuclear magnetic resonance spectroscopy (H1-NMR). The crystal is compared with the standard product of ovatodiolide by high performance liquid chromatography (HPLC) analysis, and is recognized to have ovatodiolide compounds.

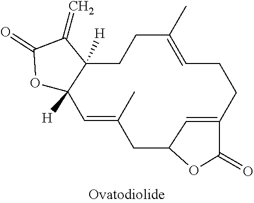 [SMILES: C=C1C(=O)O[C@H]2/C=C(\C)CC3C=C(CC/C=C(\C)CC[C@H]12)C(=O)O3]

Metabolites from Ovatodiolide (AR100-DS1):

### +O, +Cysteine: m/z:466, M2, M3, M4

### +Glutathione: m/z:636, M6, M7

### +O: m/z:345, M8, M9

| Compound | Compound name and |
|---|---|
| No. | structure |
| AR101-DS1 | 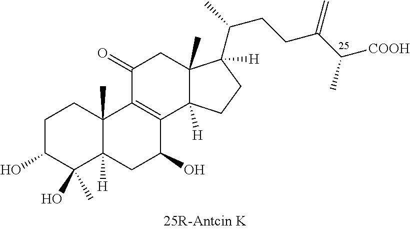 [SMILES: C=C(CC[C@@H](C)[C@H]1CC[C@H]2C3=C(C(=O)C[C@]12C)[C@@]1(C)CC[C@@H](O)[C@](C)(O)[C@@H]1C[C@@H]3O)[C@@H](C)C(=O)O] |
| | 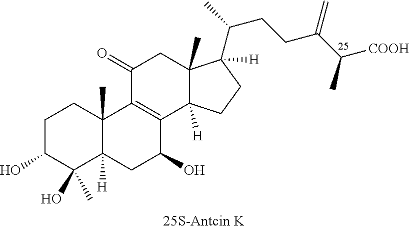 [SMILES: C=C(CC[C@@H](C)[C@H]1CC[C@H]2C3=C(C(=O)C[C@]12C)[C@@]1(C)CC[C@@H](O)[C@](C)(O)[C@@H]1C[C@@H]3O)[C@H](C)C(=O)O] |
| AR101-DS2 | 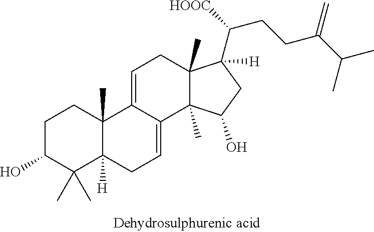 [SMILES: C=C(CC[C@@H](C(=O)O)[C@H]1C[C@H](O)[C@@]2(C)C3=CC[C@H]4C(C)(C)[C@H](O)CC[C@]4(C)C3=CC[C@]12C)C(C)C] |
| | 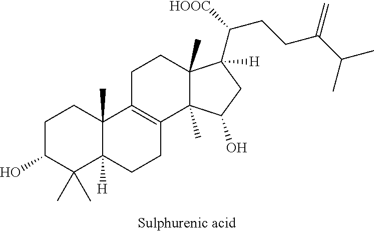 [SMILES: C=C(CC[C@@H](C(=O)O)[C@H]1C[C@H](O)[C@@]2(C)C3=C(CC[C@]12C)[C@@]1(C)CC[C@@H](O)C(C)(C)[C@@H]1CC3)C(C)C] |
| AR101-DS3 | 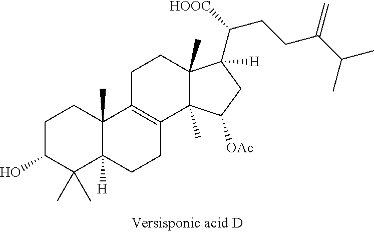 [SMILES: C=C(CC[C@@H](C(=O)O)[C@H]1C[C@H](OC(C)=O)[C@@]2(C)C3=C(CC[C@]12C)[C@@]1(C)CC[C@@H](O)C(C)(C)[C@@H]1CC3)C(C)C] |
| AR101-DS4 | 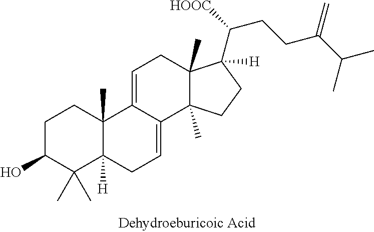 [SMILES: C=C(CC[C@@H](C(=O)O)[C@H]1CC[C@@]2(C)C3=CC[C@H]4C(C)(C)[C@@H](O)CC[C@]4(C)C3=CC[C@]12C)C(C)C] |
| AR100-DS1 | 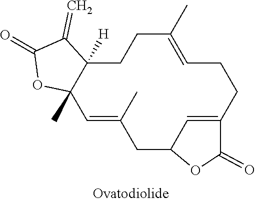 [SMILES: C=C1C(=O)O[C@@]2(C)/C=C(\C)CC3C=C(CC/C=C(\C)CC[C@H]12)C(=O)O3] |
| AR100-DS4 | 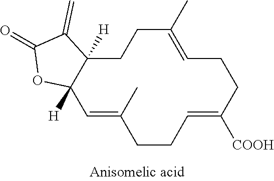 [SMILES: C=C1C(=O)O[C@H]2/C=C(\C)CC/C=C(\C(=O)O)CC/C=C(\C)CC[C@H]12] |
| AR100-DS5 | 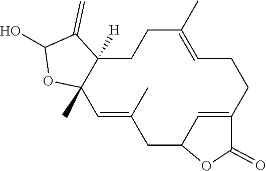 [SMILES: C=C1C(O)O[C@@]2(C)/C=C(\C)CC3C=C(CC/C=C(\C)CC[C@H]12)C(=O)O3] |
| AR100-DS6 | 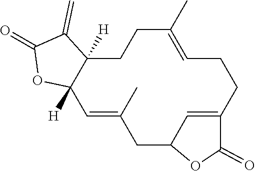 [SMILES: C=C1C(=O)O[C@H]2/C=C(\C)CC3C=C(CC/C=C(\C)CC[C@H]12)C(=O)O3] |
| AR100-DS7 | 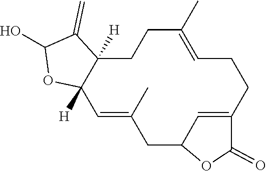 [SMILES: C=C1C(O)O[C@H]2/C=C(\C)CC3C=C(CC/C=C(\C)CC[C@H]12)C(=O)O3] |
| AR100-DS8 | 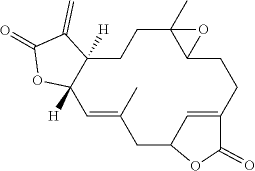 [SMILES: C=C1C(=O)O[C@H]2/C=C(\C)CC3C=C(CCC4OC4(C)CC[C@H]12)C(=O)O3] |
| AR100-DS9 | 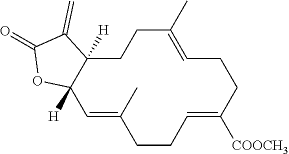 [SMILES: C=C1C(=O)O[C@H]2/C=C(\C)CC/C=C(\C(=O)OC)CC/C=C(\C)CC[C@H]12] |
| AR100-DS10 | 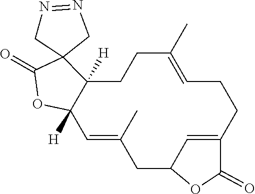 [SMILES: C/C1=C\CCC2=CC(C/C(C)=C/[C@@H]3OC(=O)C4(CN=NC4)[C@H]3CC1)OC2=O] |
| AR100-DS11 | 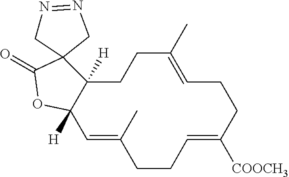 [SMILES: COC(=O)/C1=C\CC/C(C)=C/[C@@H]2OC(=O)C3(CN=NC3)[C@H]2CC/C(C)=C/CC1] |
| AR100-DS12 | 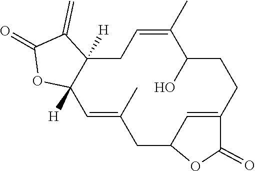 |
| AR100-DS13 | 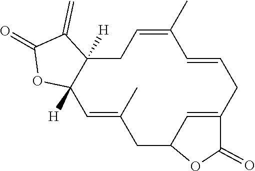 |

### Example 9. Mice Model of Cisplatin-Induced Renal Injury

Seven-to-eight-week-old male C57BL/6 mice are obtained from the BioLASCO Taiwan Co., Ltd. (Taipei, Taiwan). The animals are housed in Plexiglas cages at a constant temperature of 22±1° C. and a relative humidity of 55±5% on a 12 h dark-light cycle for at least 2 weeks before the experiment. Animals are provided food and water ad libitum. All experimental procedures are performed according to the guidelines of the Institutional Animal Ethics Committee, and the protocol is approved by the Committee for the Purpose of Control and Supervision of Experiments on Animals.

Renal fibrosis is induced via multiple injections of low-dose cisplatin. Intraperitoneal injections of cisplatin (5 mg/kg/injection; P4394, Sigma-Aldrich, St Louis, Mo.) are performed at 0, 1, and 3 weeks, for a total of 3 injections. Mice are sacrificed at 6 weeks after the first dose of cisplatin (n=6). To analyze the effects of samples, mice are given daily intraperitoneal injections for 7 days, starting from 4 weeks after first dose of cisplatin, and sacrificed at 4 weeks (n=6).

### Example 10. Antrodia camphorata Extract and Compounds Reduced Renal Dysfunction and Histopathological Changes in Cisplatin-Induced Mice

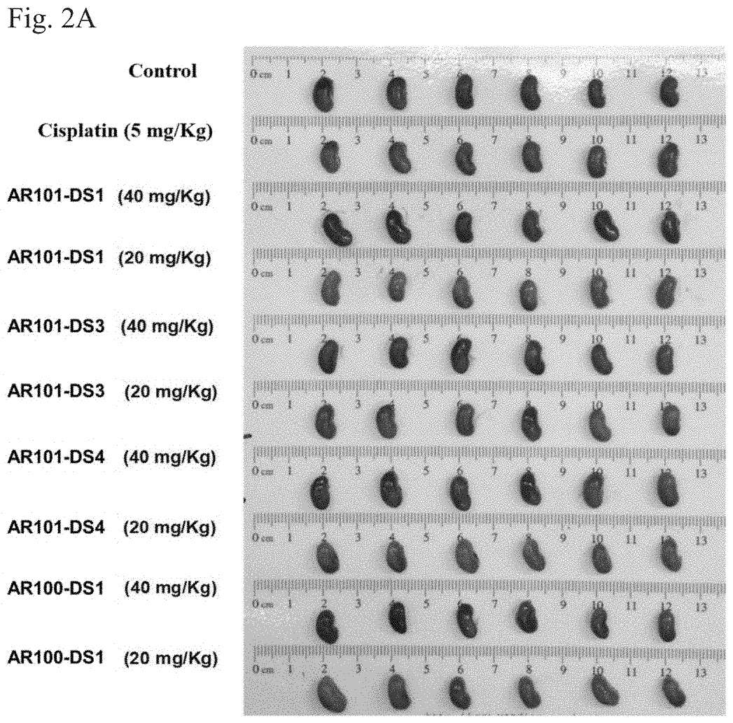

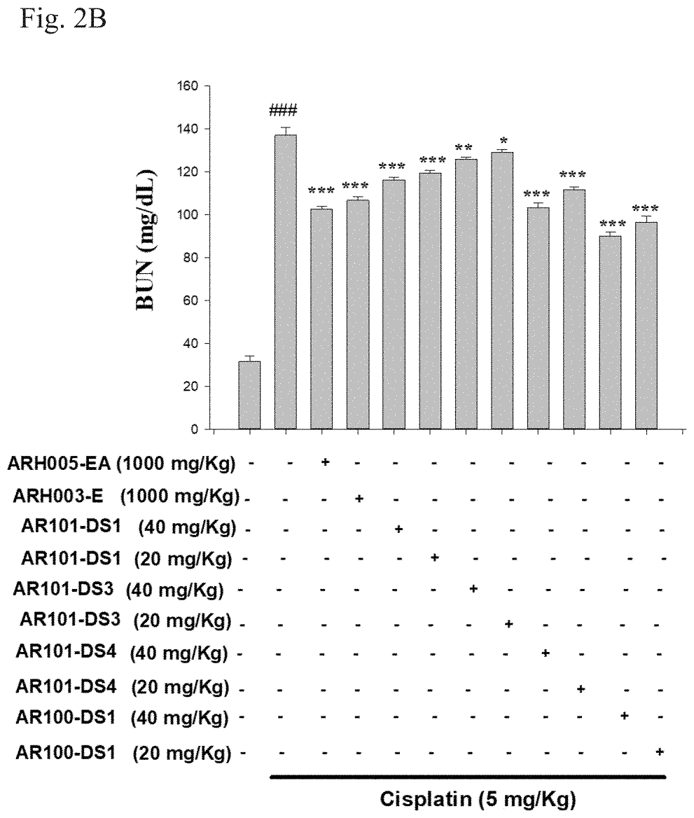

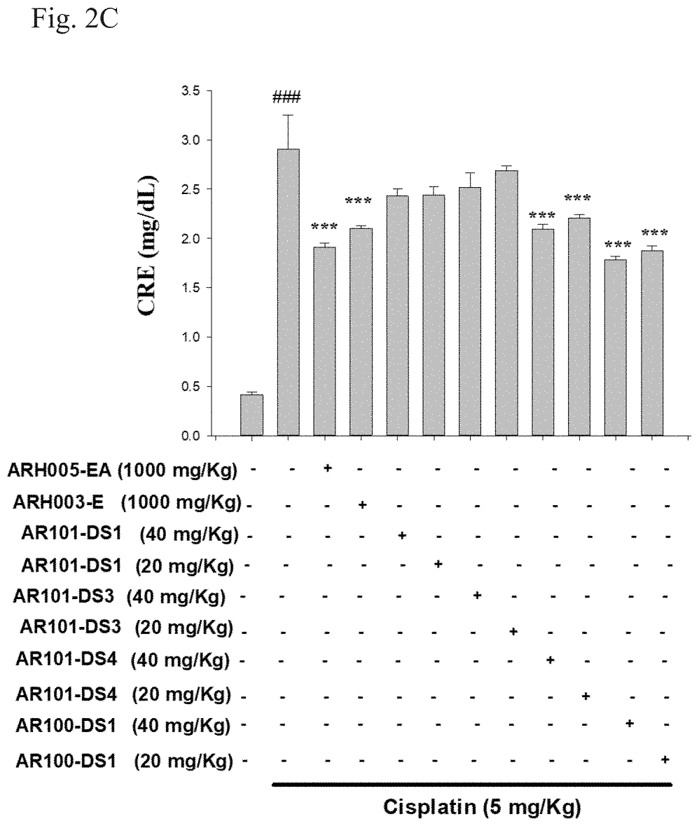

The morphological changes in the kidneys are shown in the FIG. 2A. CRE and BUN are hallmarks of kidney function. FIGS. 2B and 2C show that, compared to the control group, cisplatin injection at the three 10 mg/kg CP doses (at 0, 1, 3 weeks) highly increased the serum CRE and BUN levels (p<0.001), indicating the generation of nephrotoxicity in the cisplatin-treated mice. Treatment with ARH005-EA and ARH003-E at doses of 1000 mg/kg and compounds (AR101-DS4 and AR100-DS1) exerted a significant renal protection effect in a dose-dependent manner, as demonstrated by the normalization of CRE and BUN (p<0.001) compared to cisplatin-stimulated group.

### Example 11. Antrodia camphorata Extract and Compounds Alleviates Renal Dysfunction and Renal Injury Induced by Multiple Cisplatin Treatment

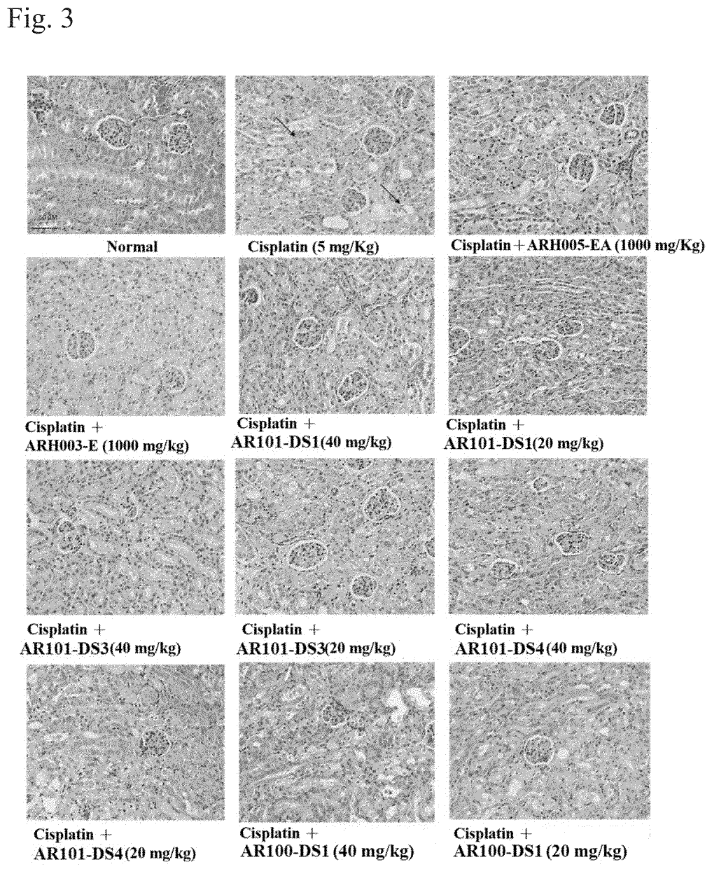

The histopathological changes are analyzed to determine whether Antrodia camphorata extract and compounds affected renal failure in cisplatin-stimulated mice. The kidney tissue of the control group is completely normal and characterized by a transparent tubular and glomerular structure with clear and normal nuclei. Kidneys had severe kidney damage in the cisplatin-stimulated mice, inducing tubular epithelial damage, inflammatory cell infiltration, tubular cell swelling, formation of intratubular casts, and tubular dilatation. However, treatment with Antrodia camphorata extract (AR005-EA) at doses of 1000 mg/kg and compounds (AR100-DS1) significantly improved necrosis and inflammatory infiltrating cells in the kidney tissue (see FIG. 3).

### Example 12. Antrodia camphorata Extract and Compounds Alerted the Cisplatin-Induced Changes in Pro-Inflammatory Cytokines and Albumin

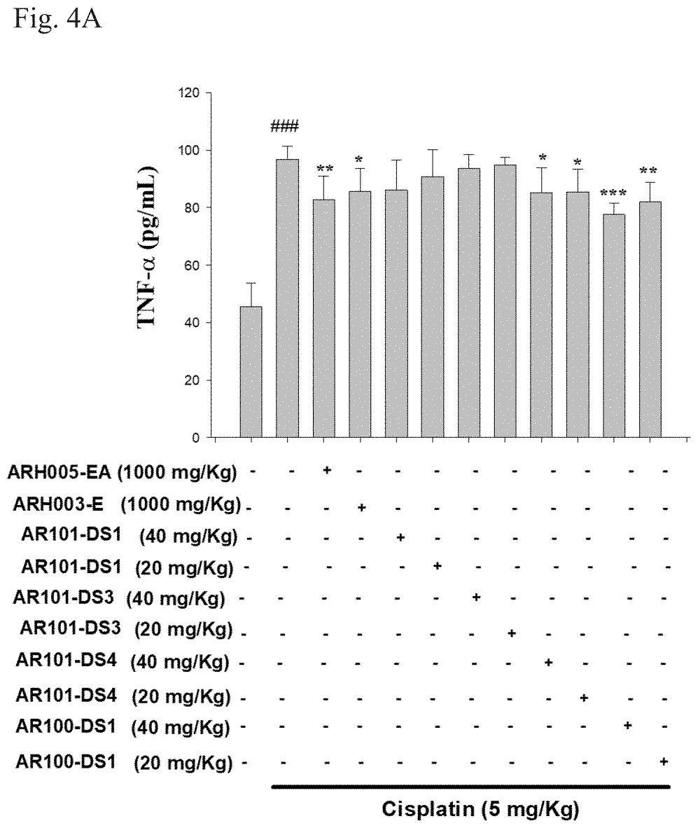

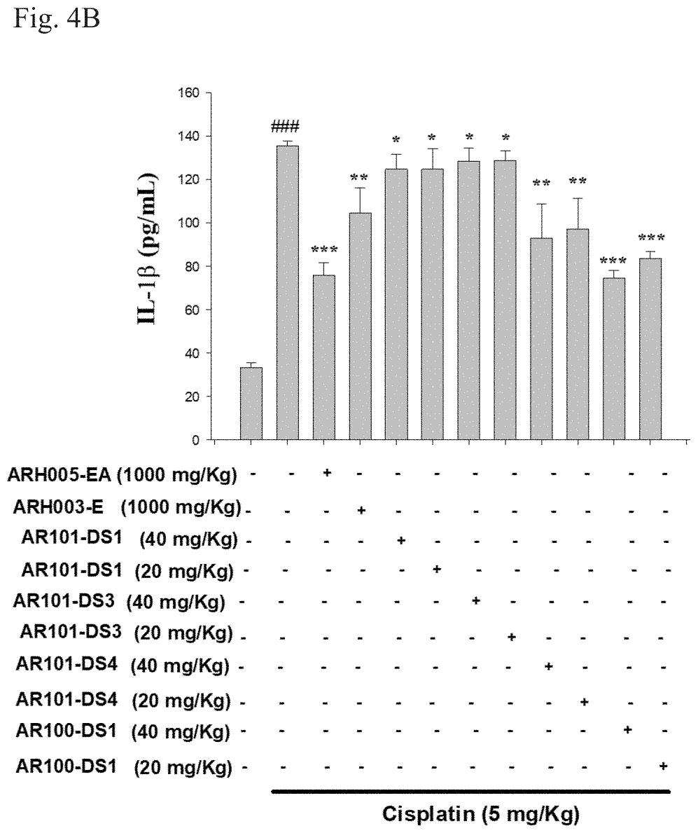

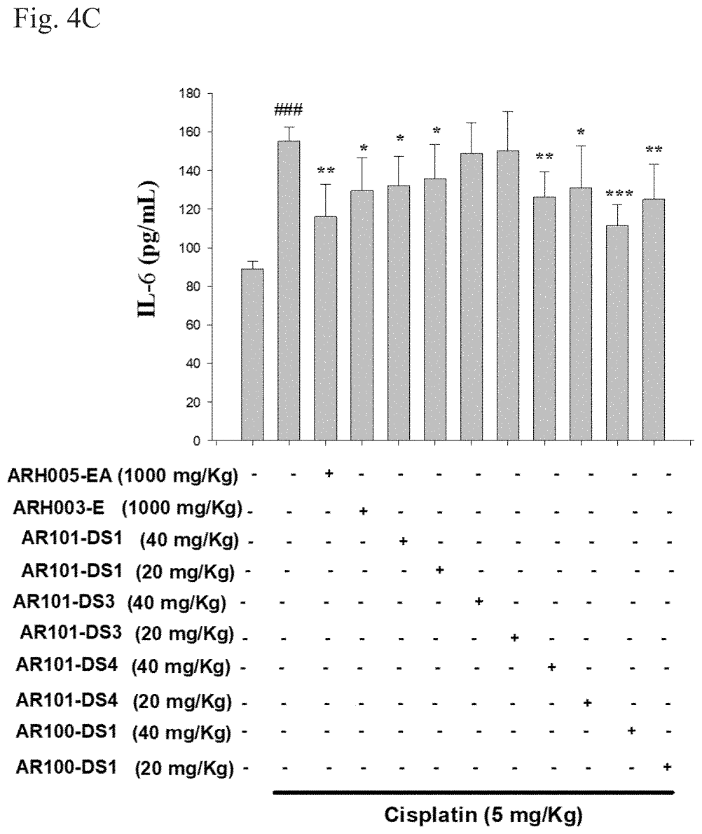

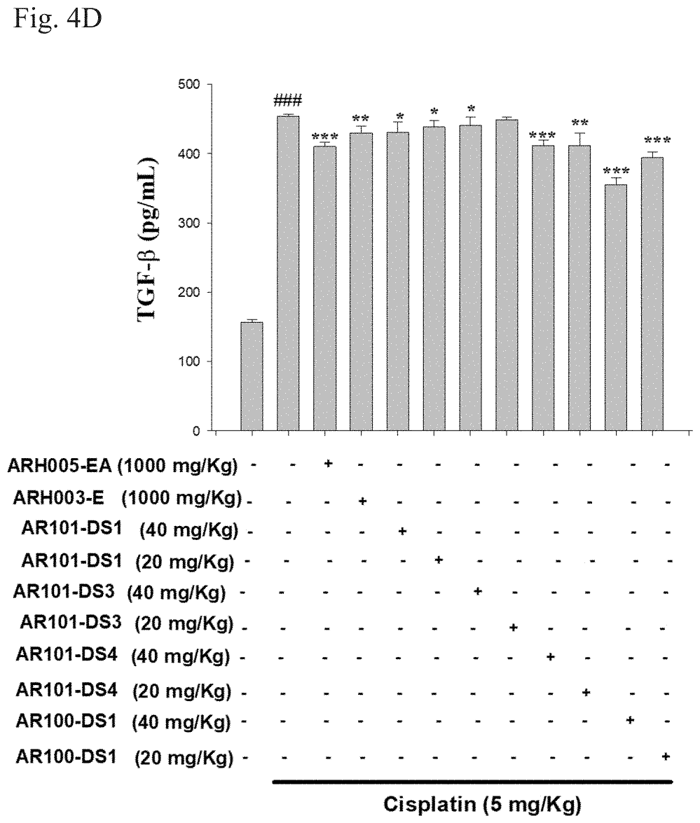

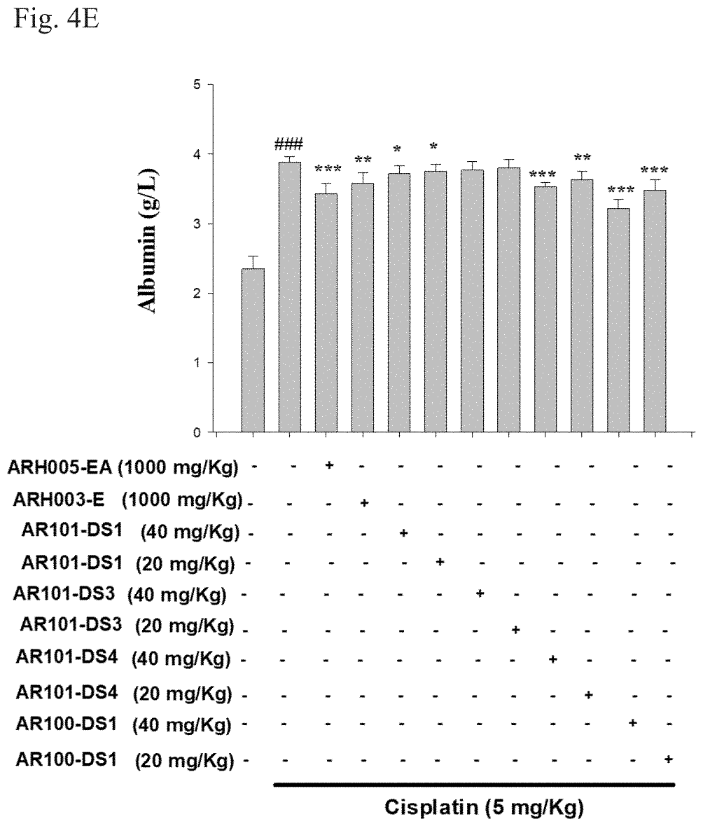

Evaluation of proinflammatory cytokine TNF-α, IL-1β, IL-6 and TGF-β levels in serum are performed by ELISA. Cisplatin-treated kidney injury mice had significantly increased NO, TNF-α, IL-1β, and IL-6 levels in serum, compared to the control group (FIGS. 4A-4E, respectively). Treatment with Antrodia camphorata Extract (AR005-EA) at doses of 1000 mg/kg and compounds (AR100-DS1) significantly improved necrosis and inflammatory infiltrating cells in the kidney tissue treatment improved NO, TNF-α, IL-1β, and IL-6 production after cisplatin challenge.

### Example 13. Inhibition of Cisplatin-Induced Renal Injury TWEAK, α-SMA, P53 and P21 Protein Expression

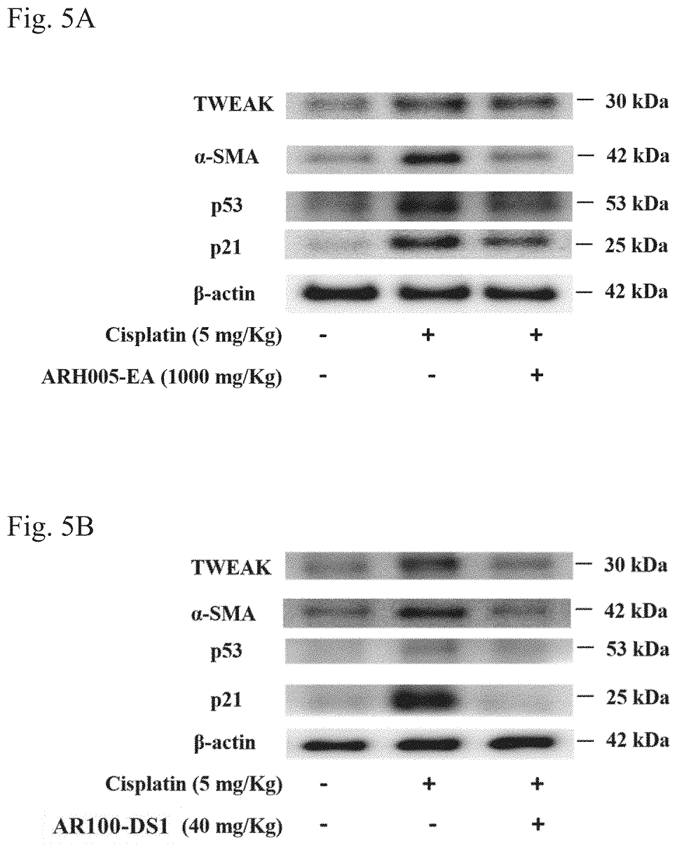

The results are examined whether pretreatment with Antrodia camphorata Extract (ARH005-EA) and compounds (AR100-DS1) inhibited cisplatin-induced TWEAK, α-SMA, P53 and P21 protein expression. The results revealed that treatment with ARH005-EA and ARH inhibited the protein expression of TWEAK, α-SMA, P53 and P21 in the kidney tissues after the cisplatin challenge (FIGS. 5A and 5B).

### Example 14. CCl₄-Induced Chronic Liver Fibrosis in Rat

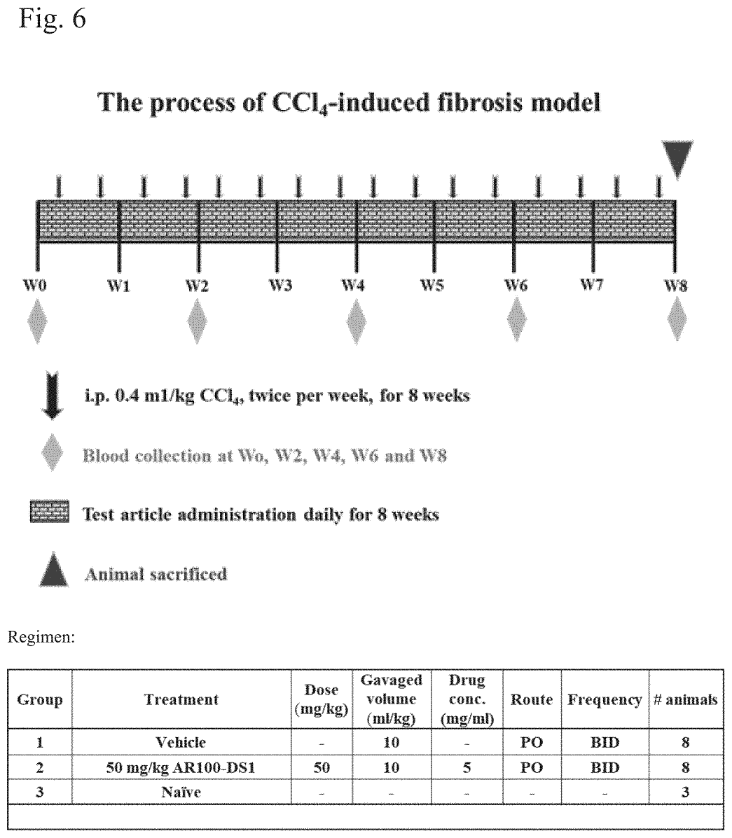

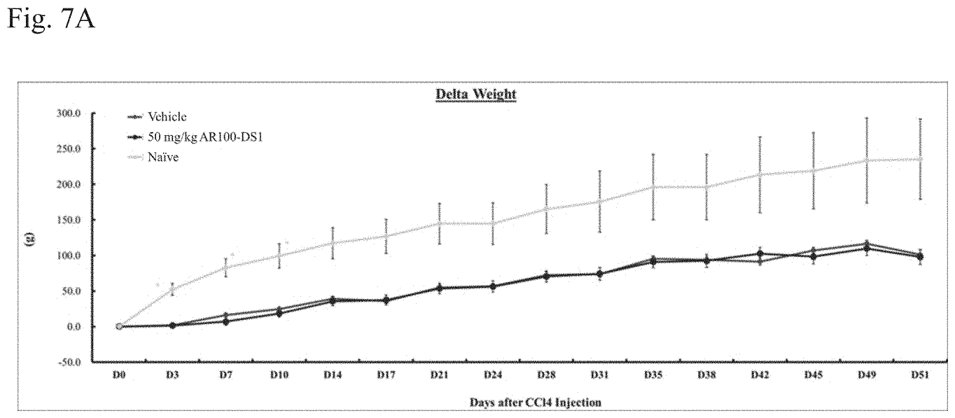

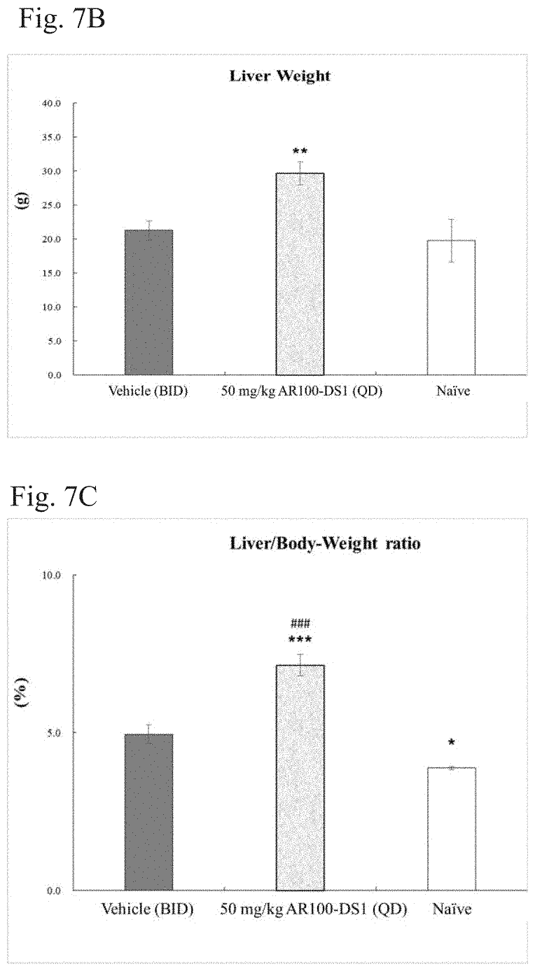

As shown in FIG. 6, eight-week-old male SD rats are administered CCl₄ at 0.4 mg/kg as twice per week for 8 weeks. Blood samples are collected at week 0, 2, 4, 6, and 8. The animals are sacrificed at the end of the 8-week for histopathological examinations. FIGS. 7A, 7B, 7C depict delta weight, liver weight, and liver/body-weight ratio, respectively. The liver weight of Naïve group is not significantly different from that of Vehicle group; however, the liver/body-weight ratio of Naïve group is significantly smaller than that of Vehicle group. The liver weight and liver/body-weight ratio of 50 mg/kg AR100-DS1 group are significantly larger comparing to that of Vehicle and Naïve group.

### Example 15. Serum Liver Enzymes Profile

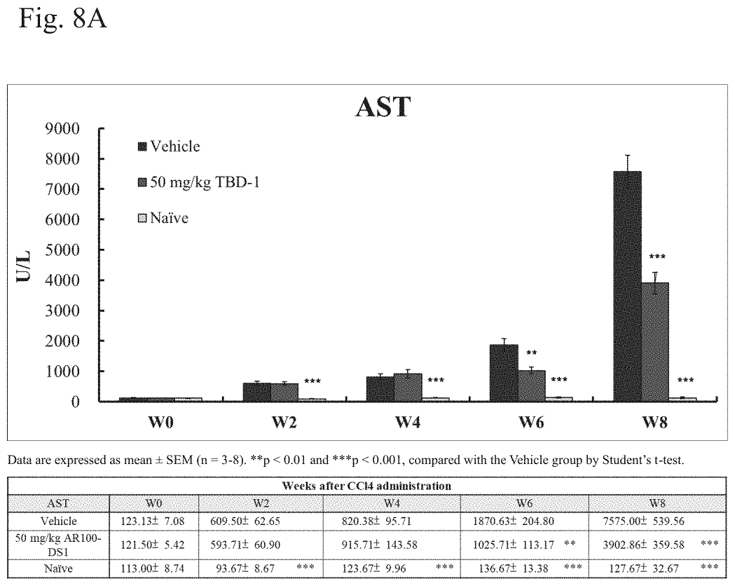

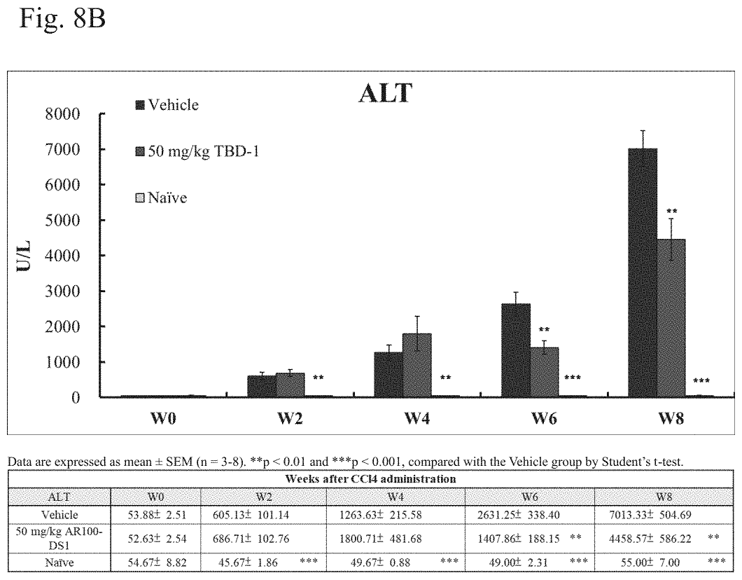

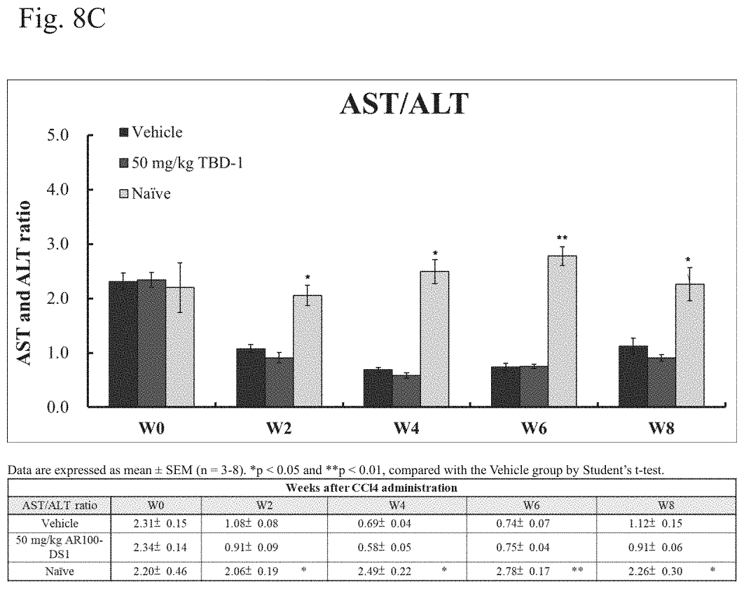

The level of clinical biochemistry such as aspartate aminotransferase (AST), alanine aminotransferase (ALT), is evaluated to determine the enzymatic activities of the livers of the control groups and the experimental groups (shown in FIGS. 8A-8C). The level of AST, ALT and AST/ALT ratio of Naïve group showed no significant changes during the experiment. The serum AST and ALT level of the animals in each test group increased significantly with the progress of the experiment; however, compared with the vehicle group, less AST and ALT increases can be observed in the 50 mg/kg AR100-DS1 group at W6 and W8.

### Example 16. Liver Histological Evaluation

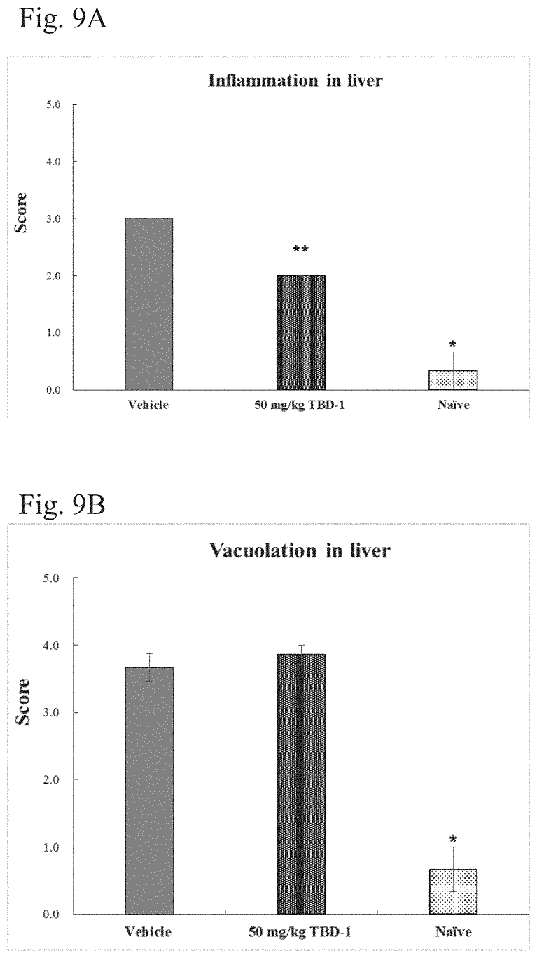

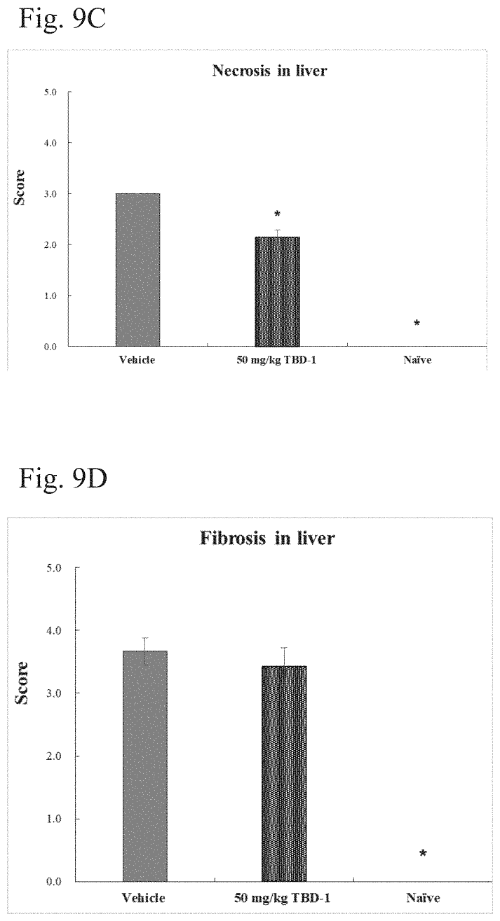

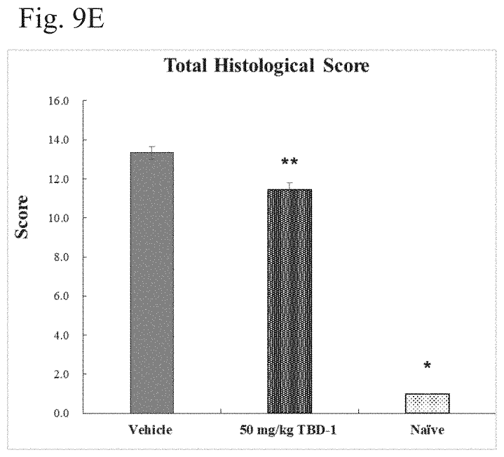

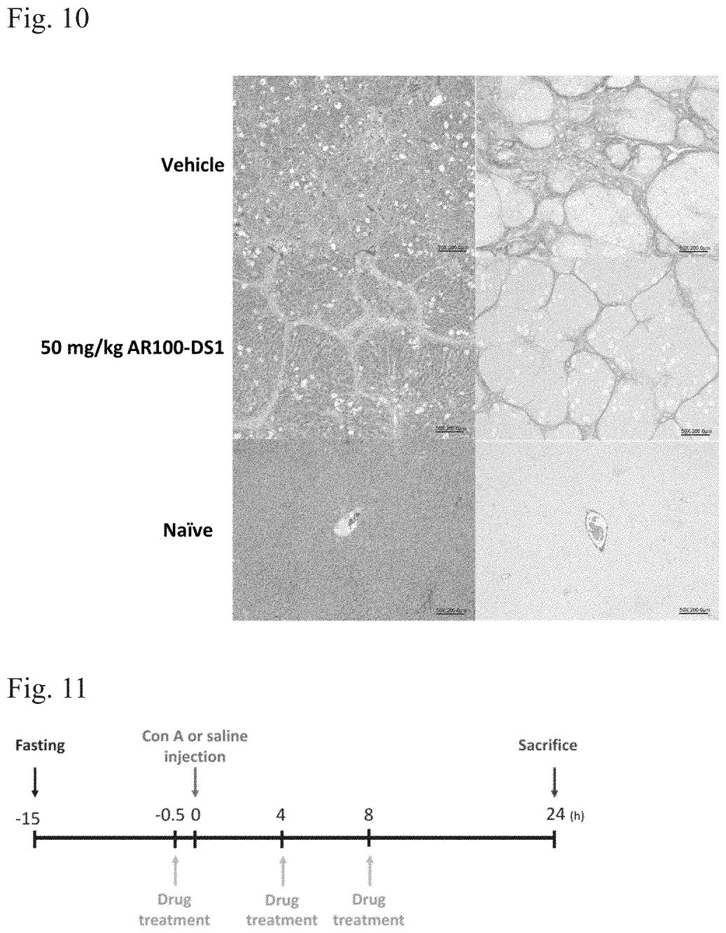

After 8 weeks of CCl₄ induction, the Vehicle group significantly suffered from liver injury such as increased AST and ALT, decreased AST/ALT ratio, inflammation, fibrosis, vacuolation and necrosis. As shown in FIGS. 9A-9E and 10, the liver of 50 mg/kg AR100-DS1 group has a smooth surface without atrophy and sclerosis, and the liver weight and liver/body weight-ratio are significantly larger than that of Vehicle and Naïve group. Overall, AR100-DS1 has been shown to have the potential to partially repair CCl₄-induced liver injury.

### Example 17. Effects of Ovatodiolide (AR100-DS1) on Con A (Concanavalin A) Induced Acute Hepatitis in BALB/c Mice

Intravenous injection of concanavalin A (Con A) is a widely used strategy to study T cell mediated hepatitis. Con A is a lectin that can activate CD4⁺ T cells, produce cytokines, and lead to liver cell damage. Dexamethasone (Dex) is a long acting synthetic corticosteroid, and has been used as anti-inflammatory and immunosuppressive medication. The effects of Ovatodiolide (AR100-DS1) on serum glutamic-pyruvic transaminase (GOT), glutamic-oxaloacetic transaminase (GPT), circulating cytokines and liver histopathology on Con A-induced acute hepatitis are evaluated in BALB/c mice.

Con A and Dex were purchased from Sigma Aldrich (USA). ProcartaPlex™ immunoassays kit was purchased from Corning Inc. (USA). GOP and GPT Fuji Dri-Chem slides were purchased from Winning Medical Inc. (Taiwan).

Male BALB/c mice (7-9 weeks old) were purchased from BioLASCO Taiwan Co., Ltd or National Laboratory Animal Center (NLAC, Taiwan). Animals are housed five per cage with food and water provided ad libitum throughout the experiments. Room temperature is maintained at 23±2° C. with an alternating 12 h light dark cycle. Animals are acclimatized for one week to minimize the effect of stress before the experiments. All experimental protocols involving animals and their care are approved by the Institutional Animal Care and Use Committee (IACUC) in ITRI (ITRI-IACUC-2018-041 and ITRI-IACUC-2018-050; accredited by AAALAC) and are carried out according to the regulations of the Council of Agriculture, Taiwan.

Con A is dissolved in pyrogen free saline at a concentration of 3 mg/mL and intravenously injected at a dose of 15 mg/kg or 20 mg/kg of body weight to induce hepatitis. Ovatodiolide (AR100-DS1) and Dex are orally administered 30 min before and then 4 h and 8 h after Con A treatment. Blood and liver tissues are collected 24 h after Con A treatment (FIG. 11). Serum are stored at 80° C. until analysis.

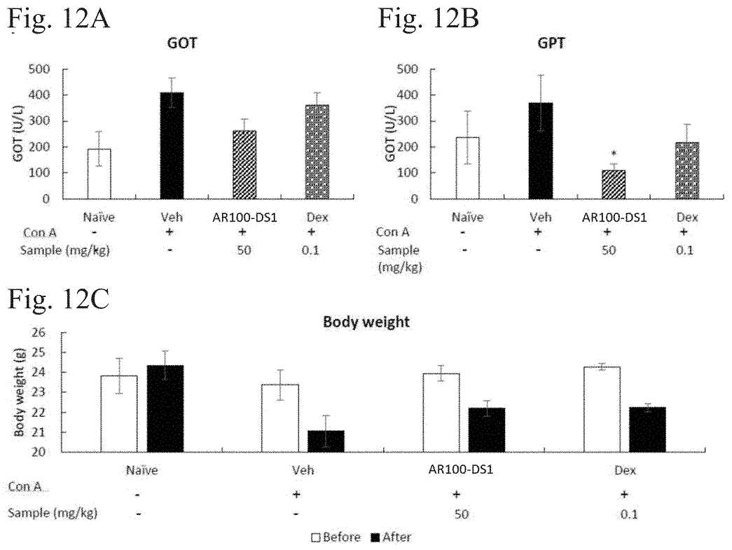

To assess the level of hepatocellular injury after Con A treatment, serum GPT and GOT levels are measured by Fuji Dri-Chem slides (Fuji, Japan). The serum of the same group is pooled for cytokine assay. Cytokine levels are measured by ProcartaPlex™ immunoassays kit according to manufacturer's instructions. Data are presented as mean±SEM. T test is used to analyze the differences between drug and vehicle treated groups. The difference is regarded statistically significant when p value is less than 0.05. Ovatodiolide (AR100-DS1) at 50 mg/kg significantly reduced GPT level that is increased by Con A (109±25 vs 368±107 U/L, p<0.05) and slightly improved elevation of GOT (261±45 vs 410±56 U/L) (FIG. 12).

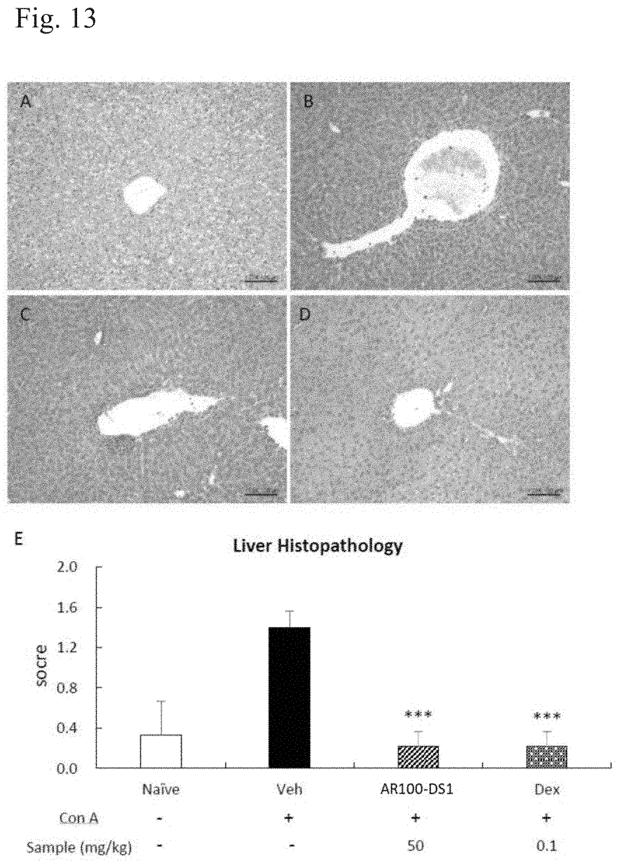

Liver tissues are fixed in 10% phosphate buffered formaldehyde, embedded in paraffin, and stained with hematoxylin and eosin (H&E) in order to confirm tissue lesions. Tissue lesions are examined microscopically by a veterinary pathologistat BioLASCO Taiwan Co., Ltd. The criteria of severity grading system for all microscopic lesions are graded from 0 to 4 as follows: 0=none; 1=individual cell necrosis; 2=≤30% lobular necrosis; 3=≤60% lobular necrosis; 4=≥60% lobular necrosis. The histopathological analysis showed Ovatodiolide (AR100-DS1) ameliorated liver necrosis (score 0.2±0.2 vs 1.4±0.2, p<0.05) (FIG. 13). The results show that ovatodiolide (AR100-DS1) reduced serum GOP and GPT and attenuated Con A induced liver necrosis.

### Example 18. Evaluation of the Efficacy of Antrodia camphorata Extract and AR101-DS2 in Preventing Atherosclerosis and Liver Fibrosis

Experimental Model

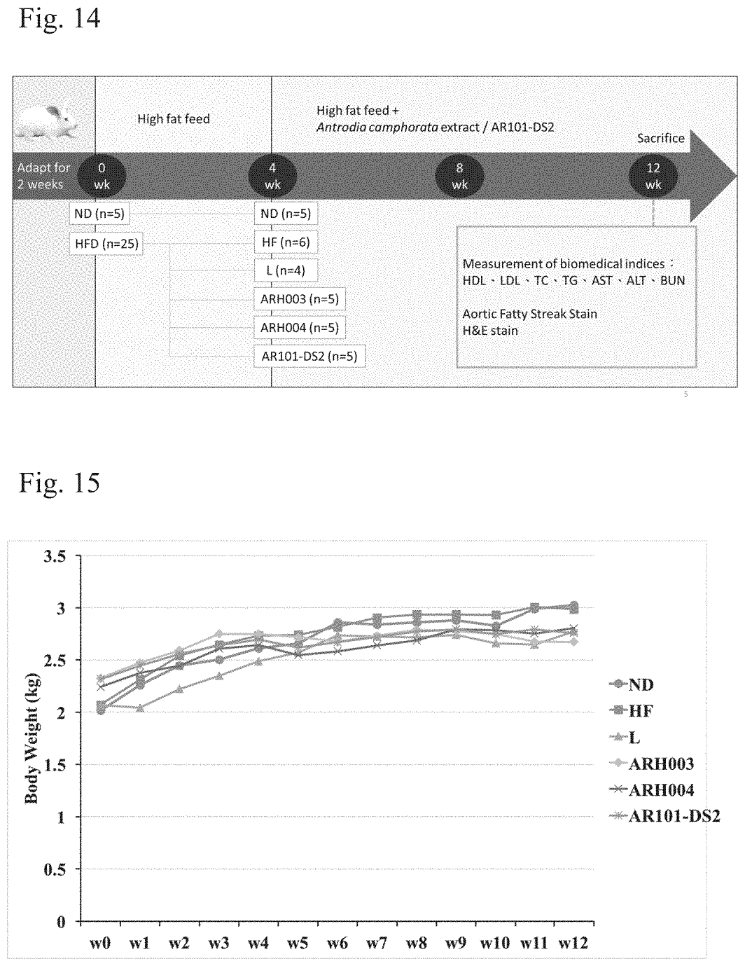

2 to 3 kg male, New Zealand White rabbits are individually caged and housed in temperature and humidity-controlled rooms. Light-dark cycles are 12 h each. After several days of acclimation, the animals are sequentially assigned to six feeding groups: standard rabbit chow, standard rabbit chow containing 0.5% cholesterol, standard rabbit chow containing both 0.5% cholesterol and 10 mg/kg Lovastatin, standard rabbit chow containing both 0.5% cholesterol and 1% ARH003, standard rabbit chow containing both 0.5% cholesterol and 1% ARH004, standard rabbit chow containing both 0.5% cholesterol and 10 mg/kg AR101-DS2. Except standard rabbit chow, others groups are given standard rabbit chow containing 0.5% cholesterol for 4 weeks (see FIGS. 14-15). The daily feeding amount for each rabbit is 50 g/kg body weight per day. Diets are administered for 8 weeks, after the animals had adjusted to their new environment. At the beginning and end of the 12 weeks study, the rabbits are anesthetized by an intramuscular injection of Zoletil 50 (1 mL/kg) (Virbac Ltd., France), and blood samples are harvested. Finally, the aortas (from aortic arch to the bifurcation of the iliac arteries) and whole livers are collected from the rabbits after they are sacrificed for further histopathological analyses.

2 to 3 kg male, New Zealand White rabbits (n=30) are divided into the following groups:

(ND) standard rabbit chow, n=5;
(HF) standard rabbit chow containing 0.5% cholesterol, n=6;
(L) standard rabbit chow containing both 0.5% cholesterol and 10 mg/kg Lovastatin, n=4;
(AR003) standard rabbit chow containing both 0.5% cholesterol and 1% ARH003, n=5;
(AR004) standard rabbit chow containing both 0.5% cholesterol and 1% ARH004, n=5;
(AR101-DS2) standard rabbit chow containing both 0.5% cholesterol and 10 mg/kg AR101-DS2, n=5; The daily feeding amount for each rabbit is 50 g/kg body weight per day.

Blood Chemistry Analysis

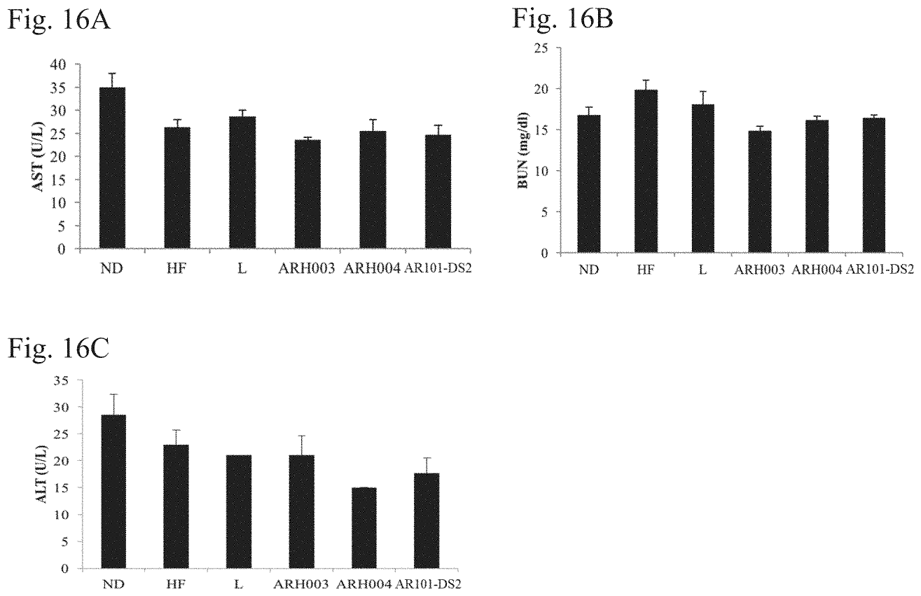

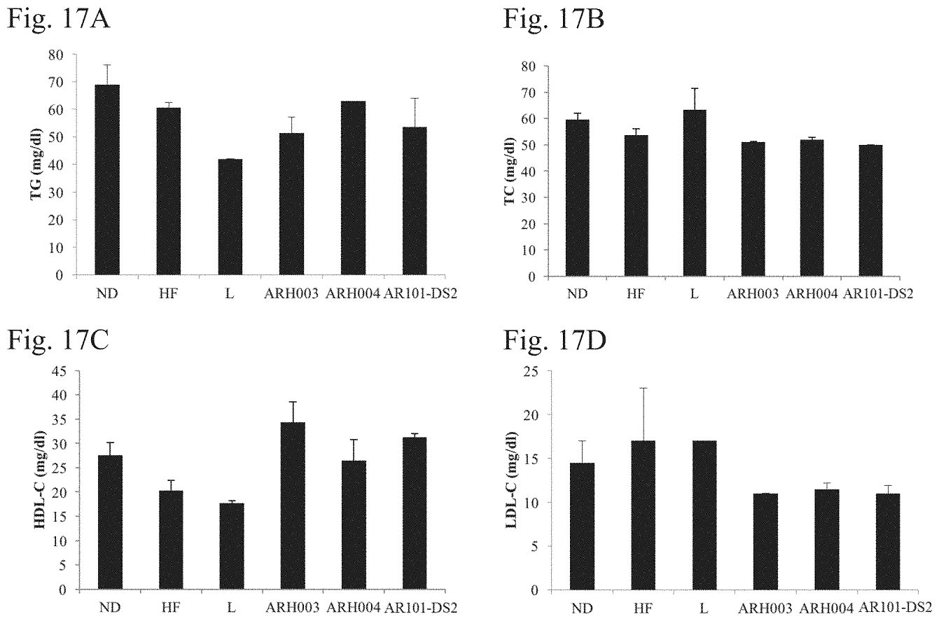

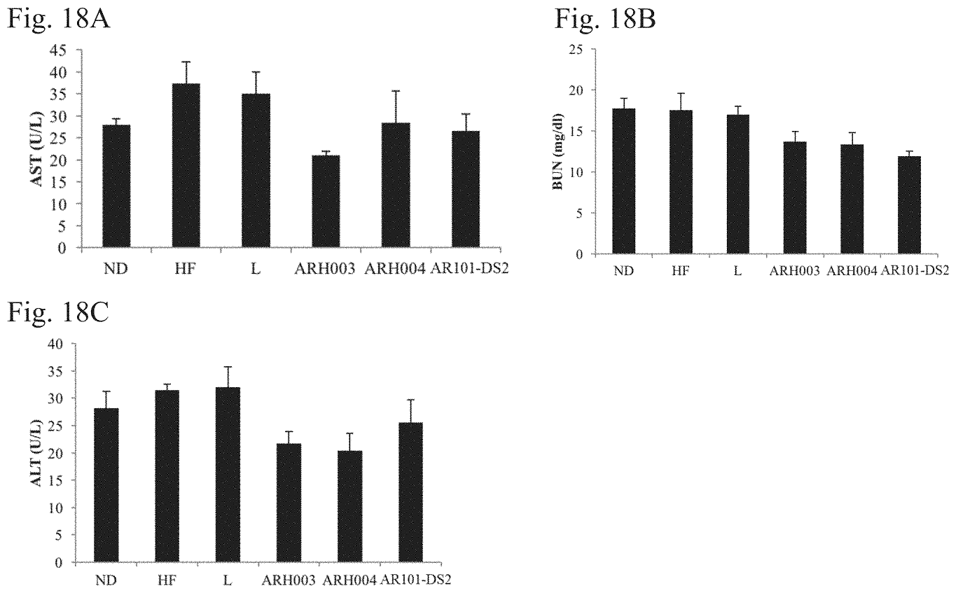

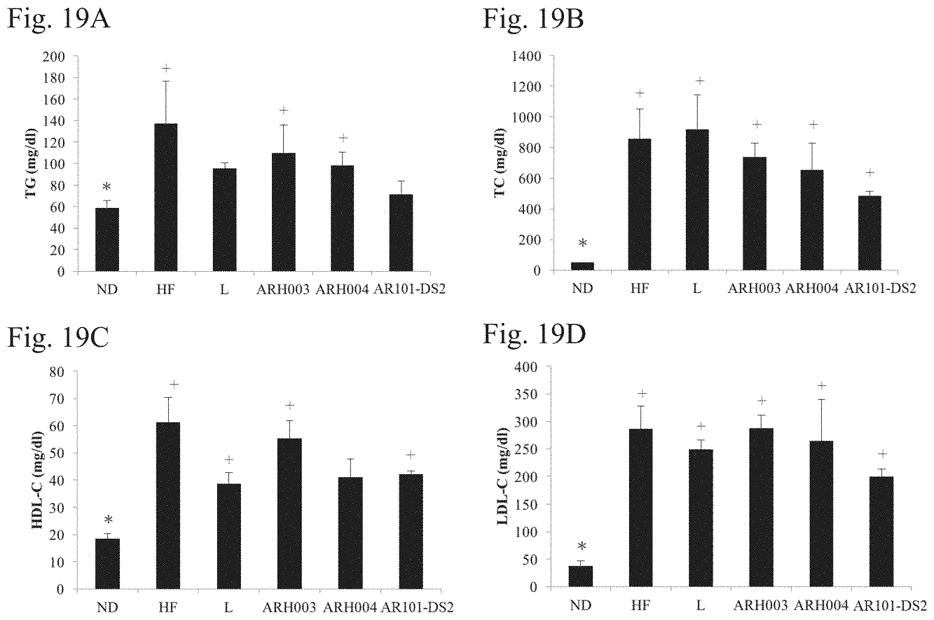

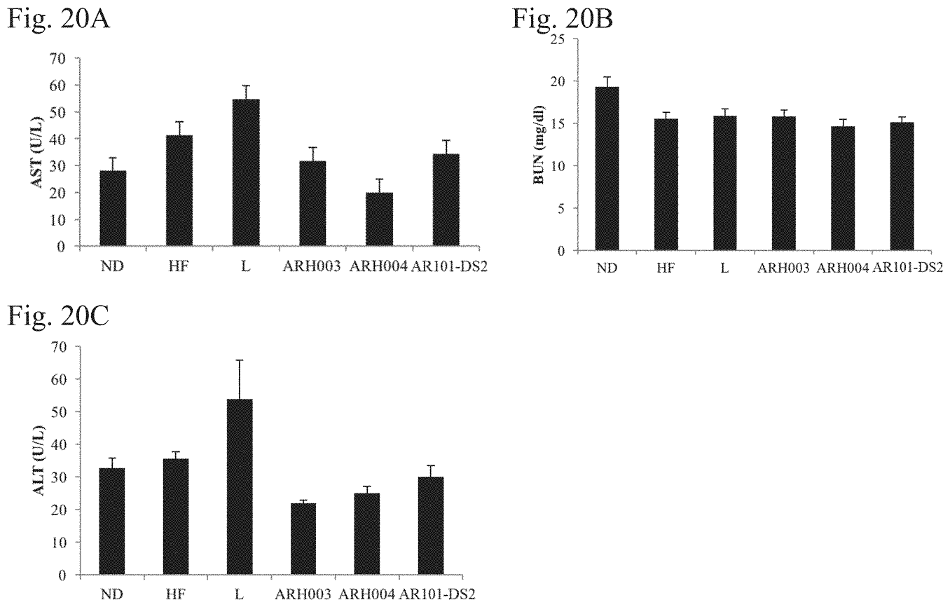

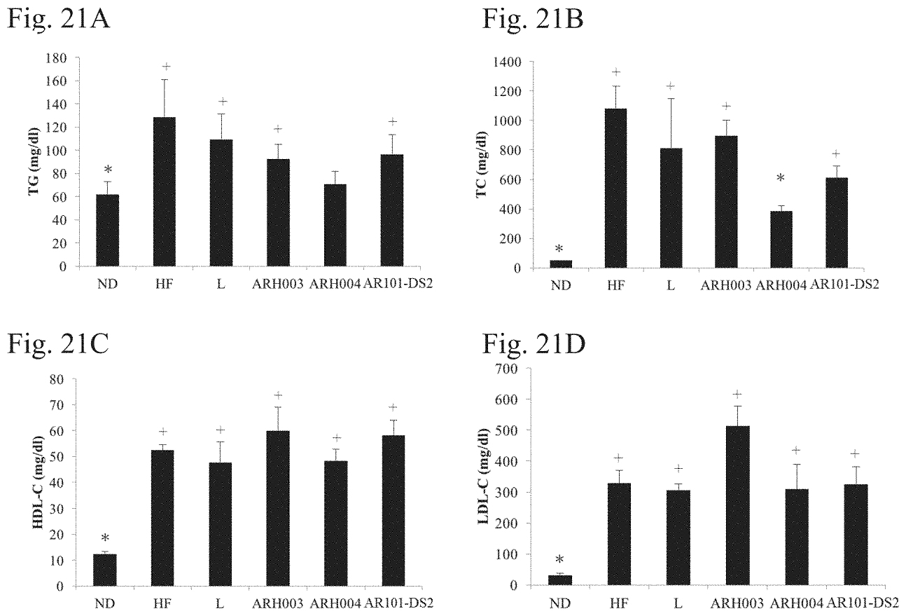

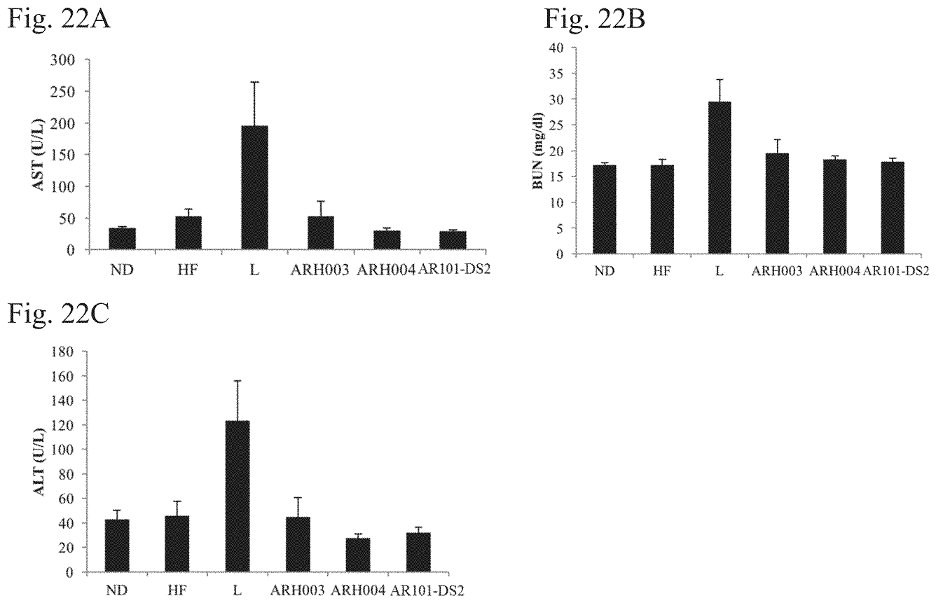

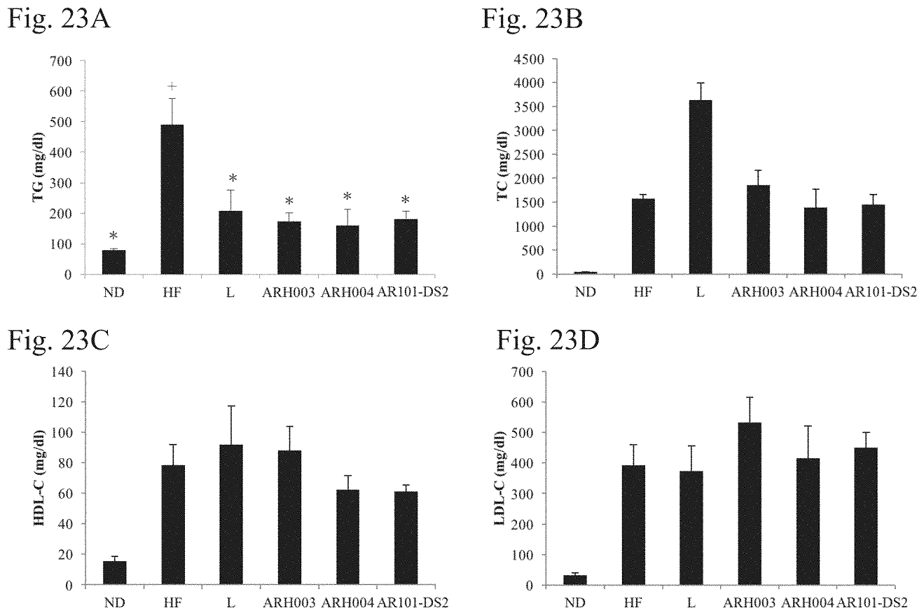

The animals are fasted overnight before blood drawing. The blood is collected from the marginal ear veins of rabbits into BD Vacutainer EDTA Blood Collection Tubes. Plasma is separated by centrifugation at 3,000 rpm at 4.0 for 10 min. FIGS. 16-23 depict measurements for changes in blood chemistry parameters included serum levels of low-density lipoprotein (LDL), cholesterol (Chol), triglycerides (TG), glutamate oxaloacetate transaminase (GOT), and glutamate pyruvate transaminase (GPT).

Aortic Fatty Streak Staining

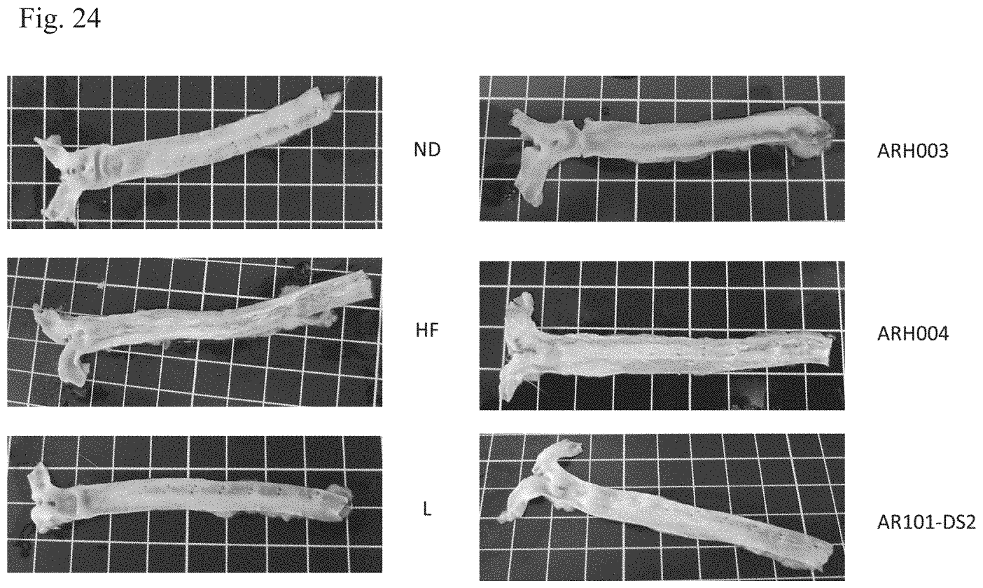

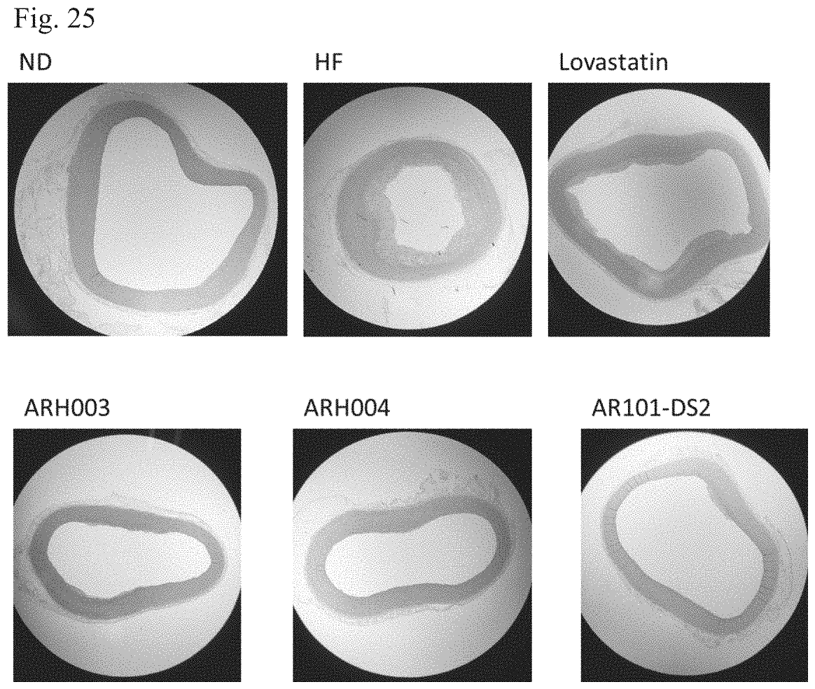

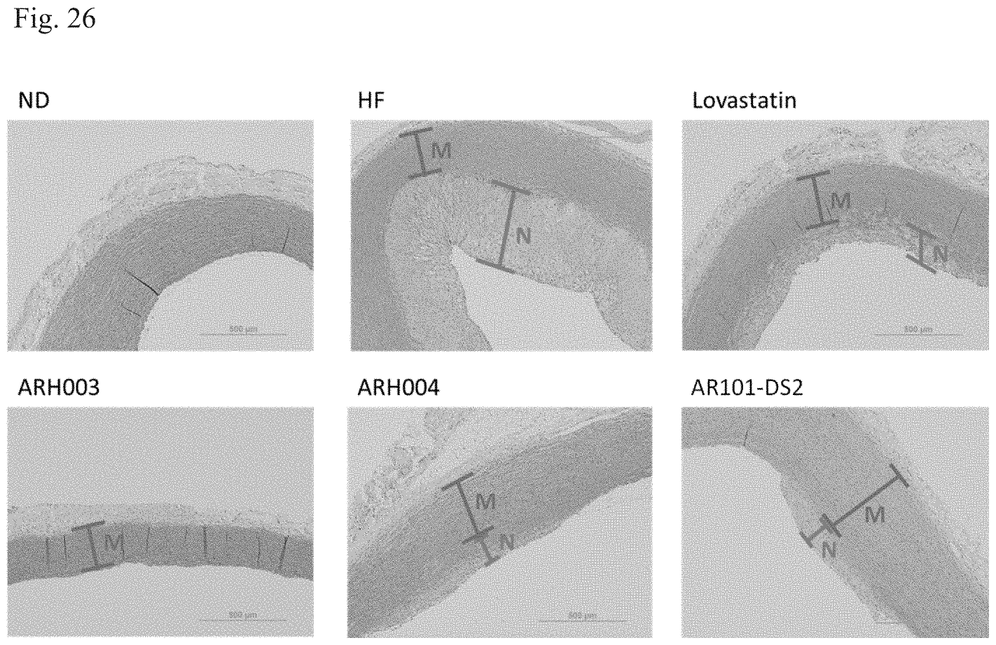

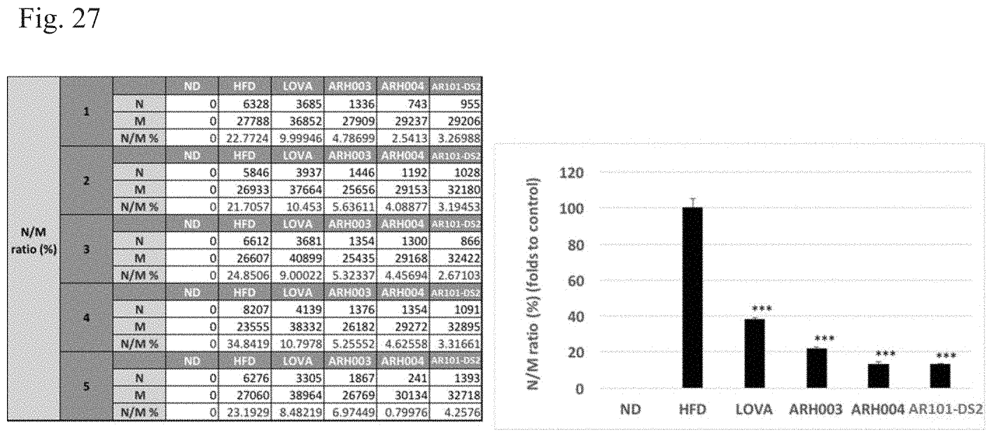

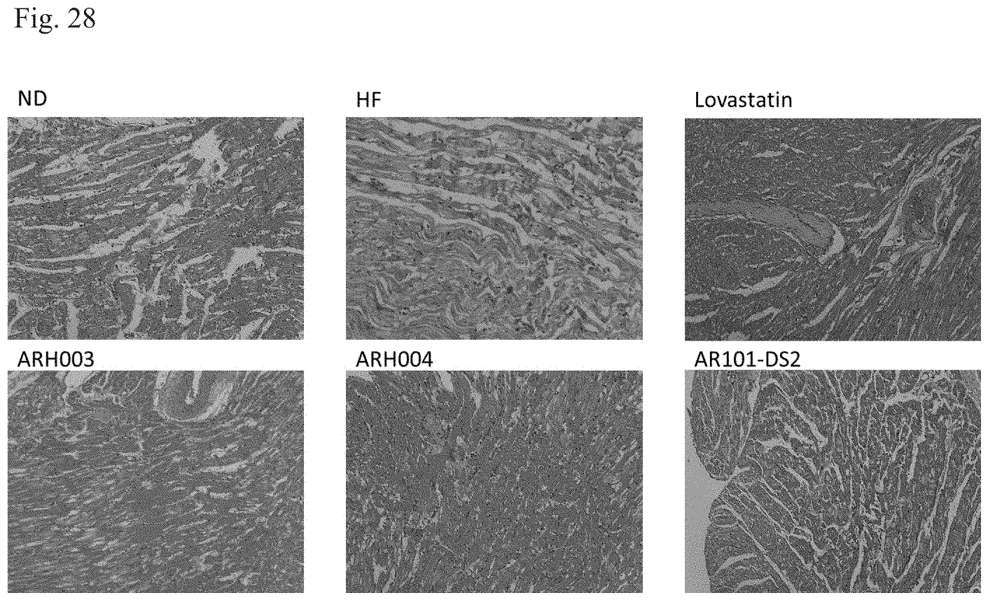

The aortas are opened longitudinally to expose the intimal surface and rinsed gently with normal saline (see FIGS. 24-26). Aortas are incubated in 2% (w/v) Sudan IV, rinsed with several concentrations (100%, 90%, 80%, 70%, and 60%) of ethanol for 1 min, and then rinsed with pure water. The photographs shown in FIG. 28 are acquired using a digital camera (Nikon D80, Japan) and quantified on an Alpha Imager 2200 documentation system (Alpha Innotech, USA). The progression of the fatty streak lesions is presented as the percentage of the stained area to the total area (FIG. 27).

Method

1. Hydrate the cells or tissue:
i. Use a microscope slide bearing cryosections or rehydrated tissue sections (see Step 12 in Cutting Sections of Paraffin Embedded Tissues) (Fischer et al. 2008) fixed in either alcohol or an aldehyde-based fixative.
ii. Immerse the slide for 30 sec with agitation by hand in H2O.
A rinse in H₂O is important; hematoxylin precipitates with salts and buffers. The staining can be performed after immunohistochemical or hybridization reactions with nonfluorescent detection systems.
2. Dip the slide into a Coplin jar containing Mayer's hematoxylin and agitate for 30 sec.
3. Rinse the slide in H₂O for 1 min.
Estimate the staining intensity at this point, and repeat Steps 2 and 3 if necessary.
4. Stain the slide with 1% eosin Y solution for 10-30 sec with agitation.
5. Dehydrate the sections with two changes of 95% alcohol and two changes of 100% alcohol for 30 sec each.
Some colorimetric substrates dissolve in alcohol.
6. Extract the alcohol with two changes of xylene.
If using plastic slides or staining in plastic culture dishes, do not use xylene or xylene-based mounting media, because they dissolve plastics.
7. Add one or two drops of mounting medium and cover with a coverslip.
If alcohols cannot be used, mount the coverslip with glycerol or other aqueous mounting media.

Reagents

Cells or tissue of interest on microscope slide (see Step 1.i)
Eosin Y (1% aqueous solution; EM Diagnostic Systems)

### Ethanol (95%, 100%)

Methanol or Flex alcohols (Richard-Allan Scientific) can be used instead of ethanol (see Step 5).

### Hematoxylin, Mayer's (Sigma)

Mayer's hematoxylin is the easiest to use and is compatible with most colorimetric substrates.
Mounting medium (Canada Balsam, Sigma C1795)
Use glycerol or other aqueous mounting media if alcohols cannot be used (see Step 7).

### Xylene

Cryosectioning of Liver Tissues

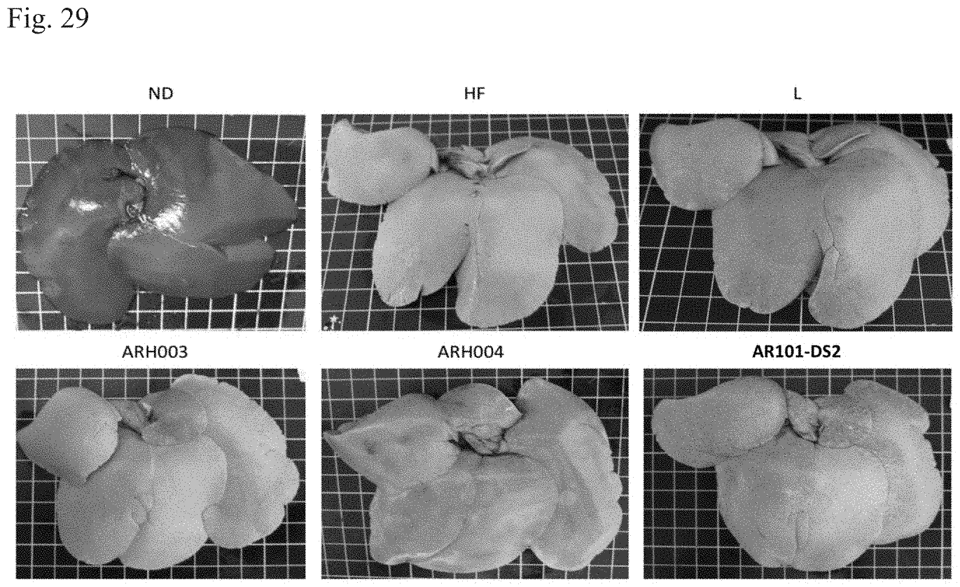

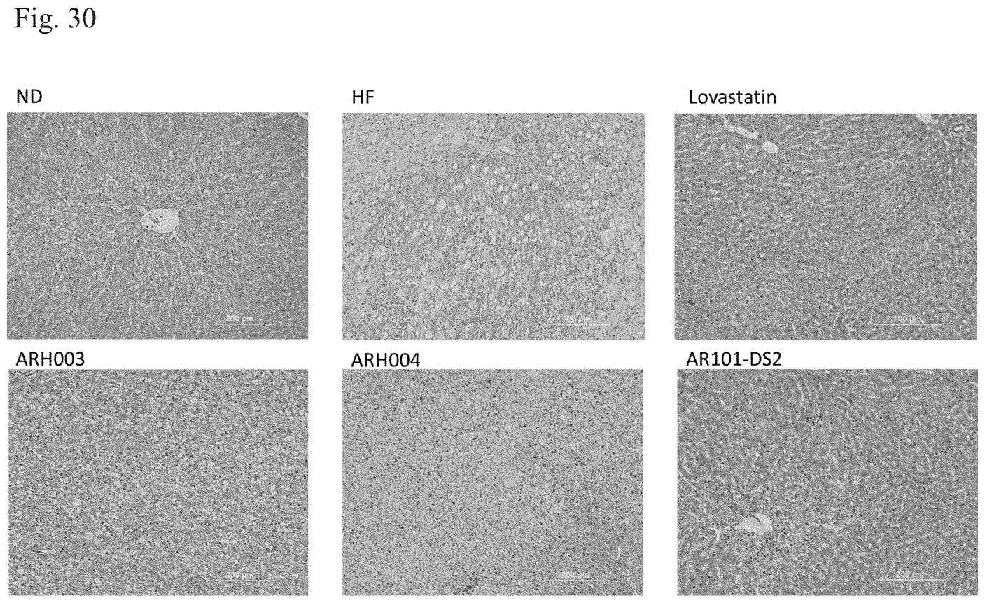

The rabbit liver tissues (shown in FIG. 29) are perfused with normal saline and fixed in 10% (v/v) formalin-neutralized solution (J.T. Baker, Inc., USA) for 24 hr. Afterward, the tissues are embedded in Tissue Tek OCT Compound (#4583; Sakura Finetek Inc., USA). Embedded tissues are cut into 10 μm thick slices and stained with Sudan IV and hematoxylin (Merck, USA). Briefly, the slices are washed with pure water for 1 min to remove the OCT compound, washed with 50% (v/v) ethanol for 30 sec, and then stained with 2% (w/v) Sudan IV for 1 hr. After further washing with 50% (v/v) ethanol and pure water for 2 min, the slices are counterstained with hematoxylin. Photographs shown in FIG. 30 are acquired using a microscope equipped with a 10-fold magnification objective and quantified on an Alpha Imager 2200 documentation system (Alpha Innotech, USA). The manifestation of fatty liver progression is presented as the percentage of the area of oil droplets to the total liver tissues (cells).

| | Score of Steatosis | | Score of | Score of |
|---|---|---|---|---|
| Slide No. | Grade | Location | Inflammation | Fibrosis |
| ND1 | 0 | 0 | 0 | 0 |
| ND2 | 0 | 0 | 0 | 0 |
| ND3 | 0 | 0 | 0 | 0 |
| HF1 | 3 | 3 | 2 | 2-3 |
| HF2 | 2 | 1-2 | 1 | 2-3 |
| HF3 | 3 | 2-3 | 1 | 3-4 |
| L1 | 2 | 1 | 2 | 1 |
| L2 | 2 | 1 | 1 | 0 |
| L3 | 0 | 0 | 0 | 0 |
| ARH003-1 | 3 | 2 | 1 | 0 |
| ARH003-2 | 3 | 2 | 1 | 0 |
| ARH003-3 | 2 | 2 | 1 | 0 |
| ARH004-1 | 3 | 2 | 1 | 1 |
| ARH004-2 | 3 | 2 | 0 | 0 |
| ARH004-3 | 3 | 2 | 1 | 0 |
| AR101-DS2-1 | 3 | 2 | 1 | 0 |
| AR101-DS2-2 | 3 | 1 | 2 | 0 |
| AR101-DS2-3 | 1 | 0 | 1 | 0 |

Score of Steatosis
Grade
0′: low- to medium-power evaluation of parenchymal involvement <5%
1′: 5-33 %
2′: 33-66%
3′: >66%
Location
0′: zone 3, centrilobular
1′: zone 2, mid-zonal
2′: zone 3, periortal
3′: panacinar
Score of Fibrosis
0′: none
1′: mild perisinusoidal or periportal
2′: perisinusoidal and portal/periportal
3′: bridging fibrosis
4′: cirrhosis
Score of Inflammation
0′: no foci
1′: mild, 2 foci per 200 field
2′: moderate, 2-4 foci per 200 field
3′: severe, 4 foci per 200 field

### Example 19. The Protective Effect of Antrodia camphorata Extract and Compounds on Bleomycin-Induced Pulmonary Fibrosis in Mice

Animals and Treatments

Specific pathogen-free ICR mice (males) (body weights 18-22 g) were purchased from the BioLASCO Taiwan Co., Ltd. (Taipei, Taiwan). The animals were housed in Plexiglas cages at a constant temperature of 22±1° C. and a relative humidity of 55±5% on a 12 h dark-light cycle for at least 2 weeks before the experiment. Animals were provided food and water ad libitum. All experimental procedures were performed according to the guidelines of the Institutional Animal Ethics Committee, and the protocol was approved by the Committee for the Purpose of Control and Supervision of Experiments on Animals.

BM-Induced PF in Mice

Mice were divided into fifth groups with 5 animals per group according to body weight: control group, BLM group, BLM+DEX group (7.5 mg/kg), BLM+ACH dosage group (50 mg/kg), and BLM+ACM dosage group (25 mg/kg), BLM+ACH dosage group (50 mg/kg), and BLM+ACM dosage group (25 mg/kg) BLM+AH dosage group (50 mg/kg), and BLM+AM dosage group (25 mg/kg) BLM+BH dosage group (50 mg/kg), and BLM+BM dosage group (25 mg/kg) BLM+CH dosage group (50 mg/kg), and BLM+CM dosage group (25 mg/kg) BLM+DH dosage group (50 mg/kg), and BLM+DM dosage group (25 mg/kg) BLM+EH dosage group (50 mg/kg), and BLM+EM dosage group (25 mg/kg) BLM. Pulmonary fibrosis (PF) was established in mice via a single intratracheal administration of BLM at 7.5 mg/kg mg/kg body weight. Different doses of samples were intragastrically administered daily for 21 days after BLM injury, and DEX was used as the positive control. Control and model groups received an equal volume of vehicle (0.9% NaCl) using the same schedule and route of administration.

Mouse body weights were recorded daily. Mice were sacrificed on the 21th day using excess chloral hydrate hydrochloride anesthesia. Blood was obtained for ELISA analyses, and whole lungs were removed and weighed. The right lungs were fixed in 10% formalin, dehydrated, and embedded in paraffin. The left lungs were used to determine hydroxyproline. The pulmonary coefficient was calculated using the following equation: lung weight/body weight×100%

Experimental Design

Male C57BL/6 mice were randomly divided into the following eight groups (n=6):

1. Group I: control;
2. Group II: mice received single intraperitoneal injection of BLM (7.5 mg/kg BW)
3. Group III: single dose (ACH, 0.5 g/kg)
4. Group IV: Single dose (ACM, 1.0 g/kg)
5. Group V: purified AR101-DS1 (50 mg/kg)
6. Group VI: purified AR101-DS1 (25 mg/kg)
7. Group VII: purified AR101-DS2 (50 mg/kg)
8. Group VIII: purified AR101-DS2 (25 mg/kg)
7. Group VII: purified AR101-DS4 (50 mg/kg)
8. Group VIII: purified AR101-DS4 (25 mg/kg)
7. Group VII: purified AR100-DS1 (50 mg/kg)
8. Group VIII: purified AR100-DS1 (25 mg/kg)
7. Group VII: purified ARH013-RA1 (50 mg/kg)
8. Group VIII: purified ARH013-RA1 (25 mg/kg)

Sampling of BALF

Under anaesthesia, BALF was performed four times through a tracheal cannula with 0.7 mL of saline. In each mouse examined, —2.5 mL (90%) of BAL fluid (BALF) was recovered. The supernatants of BALF were stored at −80° C. until used.

Lung Histopathology

The anterior portion of the right lung from each mouse was fixed in 10% formaldehyde phosphate buffer, embedded in paraffin, cut into 5 μm sections, and then treated with an hematoxylin and eosin (H&E) stain for histological examination under the light microscopy (Nikon, ECLIPSE, TS100, Tokyo, Japan). Images were captured with a digital camera (NIS-Elements D 2.30, SP4, Build 387) at an original magnification of 400×.

Assay of Hydroxyproline

The contents of hydroxyproline were analyzed in lung tissue following the instruction of hydroxyproline assay kit (Biosource International Inc., Sunnyvale, Calif., USA). The pulmonary tissues of mice were ground and homogenized with 1 ml of 6 mol/L potassium chloride solution, hydrolyzed at 95° C. for 5 hours, and the pH value was adjusted to 6.0-6.8. According to the instructions, the corresponding reagents were added to the reaction system and mixed thoroughly and then incubated for 15 minutes at 60° C. After cooling, the supernatants were collected after centrifuging at 3500 rpm for 10 minutes. The absorbance value of the supernatant from the samples was measured at 550 nm by a spectrophotometer and calculated for the contents of hydroxyproline on each group.

TNF-α, IL-6, and IL-1β cytokines in serum

The serum concentration of the pro-inflammatory cytokines (i.e. tumor necrosis factor-α (TNF-α), interleukin-6 (IL-6), and IL-1β) in serum were assessed with relevant enzyme-linked immunosorbent assay (ELISA) kits (Biosource International Inc., Sunnyvale, Calif., USA), based on the manufacturer's instructions.

Myeloperoxidase (MPO) Assay

The pulmonary MPO activity was a reliable index for estimating the infiltration of inflammatory cells in lungs. The lung tissues were homogenized and the MPO levels were detected with the kits according to manufacturer's instruction.

Histopathological Analyses

The right lungs were embedded in paraffin wax, fixed in 10% formalin, and processed into sections. The sections were stained with haematoxylin and eosin (H&E) or subjected to Masson's trichrome staining.

Statistical Analysis

Data obtained from animal experiments were expressed as the means and standard errors of the means (±S.E.M.). Student's t-test were used to examine the differences among multiple groups or between two groups. Statistical significance is expressed as * p<0.05, ** p<0.01 and *** p<0.001.

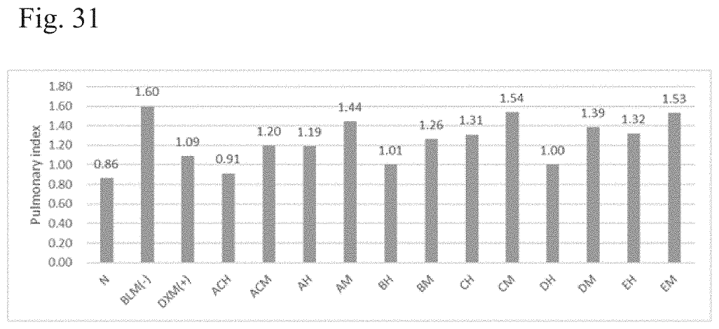

At the end point of the entire experiment, the animal's body weight and lung weight were recorded. Compared with control animals, the body weight changes of bleomycin (BLM)-administered animals were significantly reduced. Compared with other experimental groups, the lung index [(lung weight/body weight)×100] showed a significant increase in bleomycin-administered animals (Table 1 and FIG. 31). There was a significant decrease in the lung index of ACH, BH and DH.

TABLE 1
Lung index of A. camphorata Extract and Compounds on bleomycin-
induced lung fibers.

| | Initial body | Final body | Pulmonary | Pulmonary |
|---|---|---|---|---|
| | mass/g | mass/g | mass/g | index |
| Normal | 34.18 ± 0.39 | 39.73 ± 0.38 | 0.34 ± 0.01 | 0.86 ± 0.02 |
| BLM | 34.28 ± 0.39 | 35.45 ± 0.28 | 0.57 ± 0.01 | 1.60 ± 0.02*** |
| DEX | 34.24 ± 0.5 | 38.50 ± 0.22 | 0.42 ± 0.01 | 1.09 ± 0.02*** |
| ACH(1.0 g/kg) | 34.28 ± 0.44 | 39.48 ± 0.24 | 0.36 ± 0.01 | 0.91 ± 0.02** |
| ACM (0.5 g/kg) | 34.35 ± 0.45 | 38.42 ± 0.27 | 0.46 ± 0.01 | 1.20 ± 0.02*** |
| AH(AR101-DS1) (50 mg/kg) | 34.22 ± 0.39 | 39.02 ± 0.37 | 0.46 ± 0.01 | 1.19 ± 0.01** |
| AM(AR101-DS1) (25 mg/kg) | 34.35 ± 0.48 | 36.45 ± 0.28 | 0.52 ± 0.01 | 1.44 ± 0.04*** |
| BH(AR101-DS2) (50 mg/kg) | 34.44 ± 0.35 | 39.7 ± 0.21 | 0.39 ± 0.01 | 1.01 ± 0.02** |
| BM(AR101-DS2) (25 mg/kg) | 34.64 ± 0.36 | 37.91 ± 0.30 | 0.48 ± 0.01 | 1.26 ± 0.03*** |
| CH(AR101-DS4) (50 mg/kg) | 34.50 ± 0.37 | 37.47 ± 0.22 | 0.49 ± 0.01 | 1.31 ± 0.01** |
| CM(AR101-DS4) (25 mg/kg) | 34.52 ± 0.29 | 36.44 ± 0.28 | 0.52 ± 0.01 | 1.54 ± 0.04* |
| DH(AR100-DS1) (50 mg/kg) | 34.43 ± 0.25 | 39.12 ± 0.30 | 0.39 ± 0.01 | 1.00 ± 0.02** |
| DM(AR100-DS1) (25 mg/kg) | 34.50 ± 0.33 | 37.33 ± 0.25 | 0.52 ± 0.02 | 1.39 ± 0.06*** |
| EH(ARH013-RA1) (50 mg/kg) | 34.28 ± 0.29 | 37.00 ± 0.42 | 0.49 ± 0.02 | 1.32 ± 0.06** |
| EM(ARH013-RA1) (25 mg/kg) | 34.44 ± 0.31 | 35.86 ± 0.25 | 0.55 ± 0.01 | 1.53 ± 0.03** |

### Example 20. Antrodia camphorata Extract and Compounds Reduced Lung Dysfunction and Histopathological Changes in BLM-Induced Mice

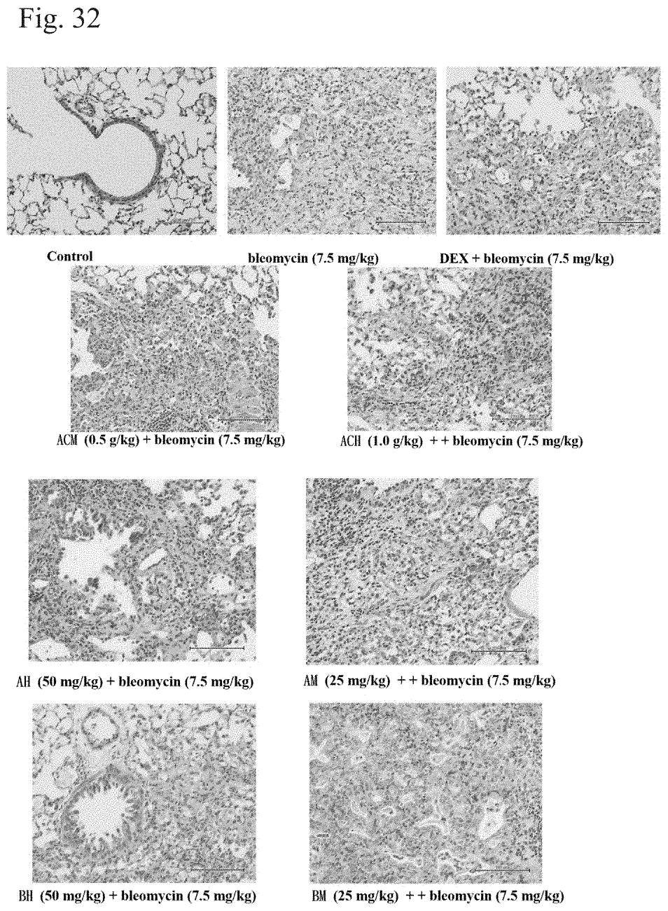

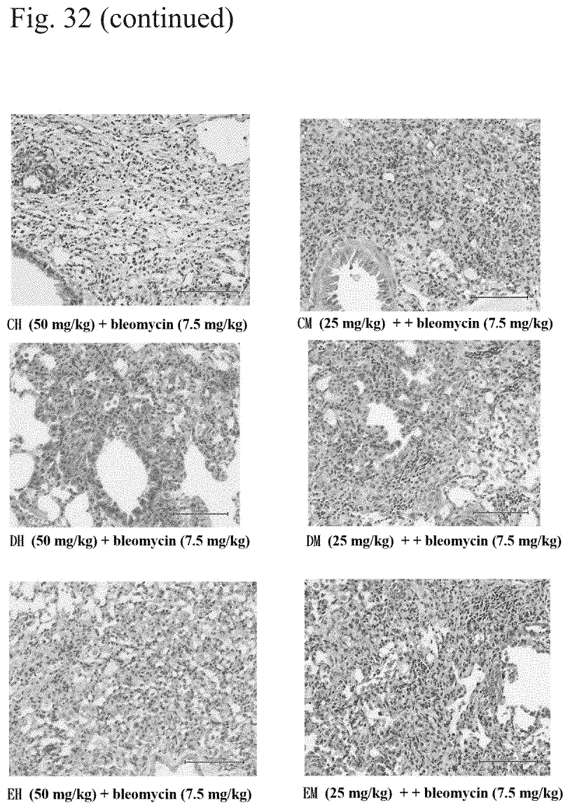

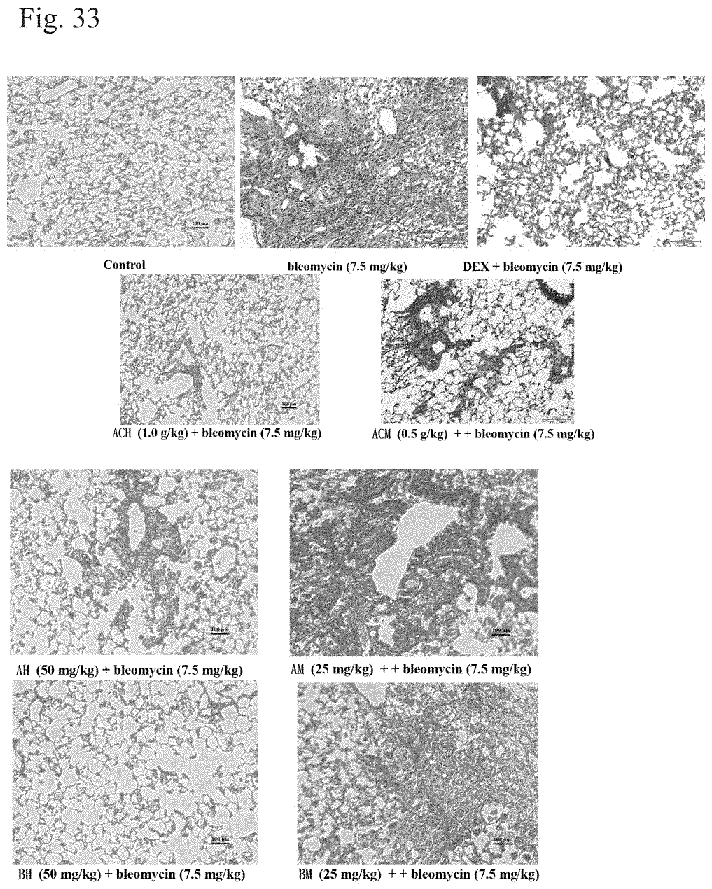

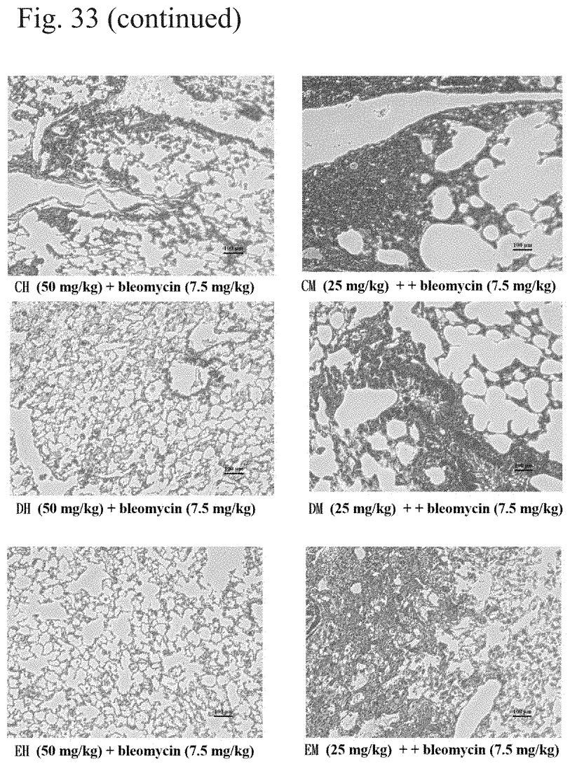

We evaluated the histopathological lung alterations in mice for exploring the therapeutic effect of A. camphorata extract and compounds. Inflammatory infiltration and integrity of organizational structures were observed by H&E staining (FIG. 32); the fibrosis degree of lung tissue was performed by Masson staining (FIG. 33). The control group demonstrated some histological findings such as a thin alveolar wall, intact alveolar structure, normal alveolar septum, and less inflammatory cells infiltration in the pulmonary mesenchyme. After 21 days of BLM administration, alveolar edema, a significant increase in septum width and increased inflammatory cells infiltration were observed. Administration of A. camphorata extract and compounds ameliorated the inflammatory infiltration and the damaged structure in lung tissue as compared to that of the BLM group.

Masson staining extensive stained blue in the lung tissue and septum after 21-day post BLM administration, suggesting a severe degree of pulmonary fibrosis in BLM group than the normal group. After A. camphorata extract and compounds treatment, the blue area was decreased, and the fibrosis degree was alleviated. At 21 days after BLM modeling, the scores of alveolitis and fibrosis were significantly decreased after A. camphorata extract and compounds therapy. The above results suggested that A. camphorata extract and compounds alleviated the degree of inflammation and fibrosis in the lungs of mice with pulmonary fibrosis.

### Example 21. Pulmonary Fibrotic Markers

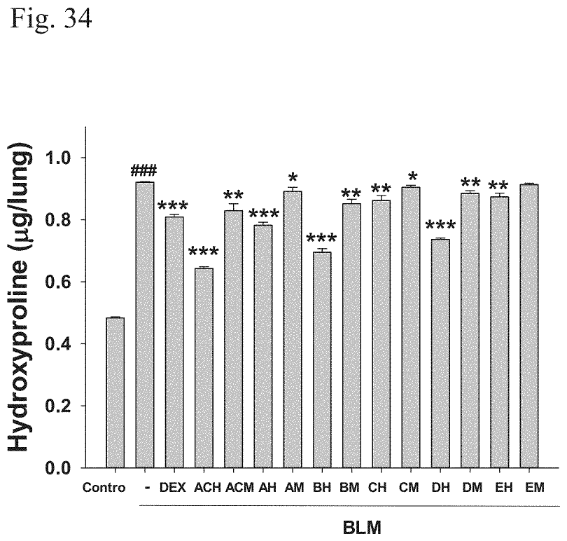

Hydroxyproline content is an important index for collagen deposition in the lung tissue. To quantify the extent of pulmonary fibrosis, the hydroxyproline content in lung tissue was measured in each group and is shown in FIG. 34. BLM obviously increased HP content (p<0.001) compared with the control group. A. camphorata Extract (1.0 g/kg) and AH, BH and DH reduced lung HP significant recovery (p<0.001).

### Example 22. Antrodia camphorata Extract and Compounds Alerted the Bleomycin-Induced Changes in Pro-Inflammatory Cytokines

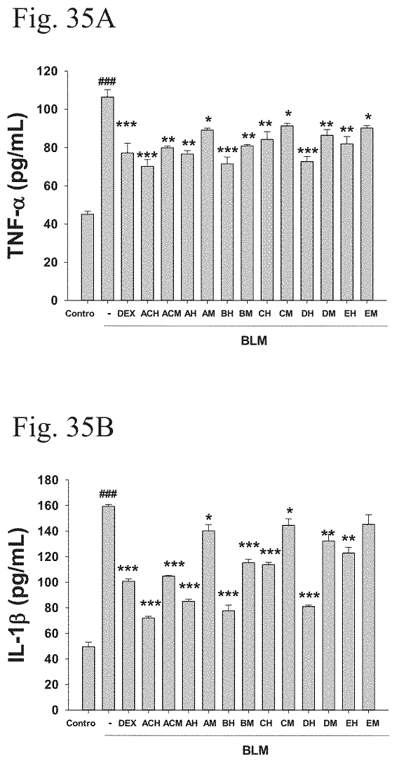

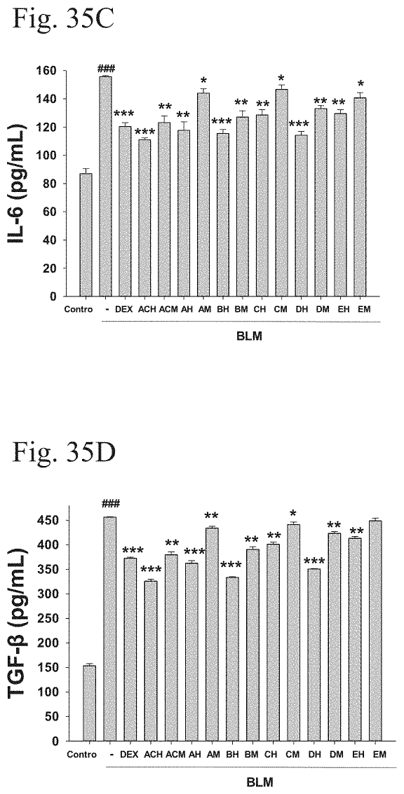

Evaluation of proinflammatory cytokine TNF-α, IL-1β, IL-6 and TGF-β levels in serum were performed by ELISA. BLM-treated kidney injury mice had significantly increased NO, TNF-α, IL-1β, and IL-6 levels in serum, compared to the control group (FIGS. 35A-34E, respectively). treatment with A. camphorata Extract at doses of 1.0 g/kg and compounds (BH and DH) significantly improved necrosis and inflammatory infiltrating cells in the lung tissue treatment improved TNF-α, IL-1β, IL-6 and TGF-β production after BLM challenge (p<0.001).

### Example 23. Effects of A. camphorata Extract and Compounds on Lung MPO Activity

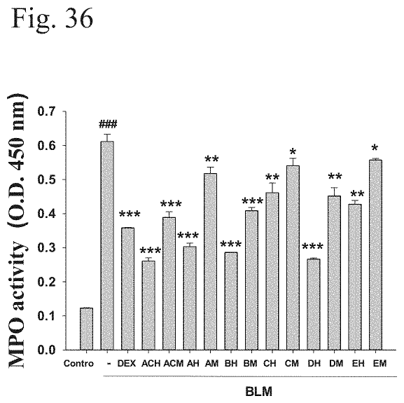

As depicted in FIG. 36, there was a remarkable increase of the MPO level in response to BLM challenge compared with that in control group (p<0.01). On the contrary, the administration of both A. camphorata extract, AH, BH, DH and Dex evidently suppressed the MPO activities compared with that in BLM group (p<0.001), which exerted stronger effect than that in A. camphorata extract and compounds group (p<0.05) (FIG. 36).

### Example 24. Effects of a Combination of A. camphorata Extract and Ovatodiolide on Oleic Acid-Induced Steatosis in HepG2 Cells

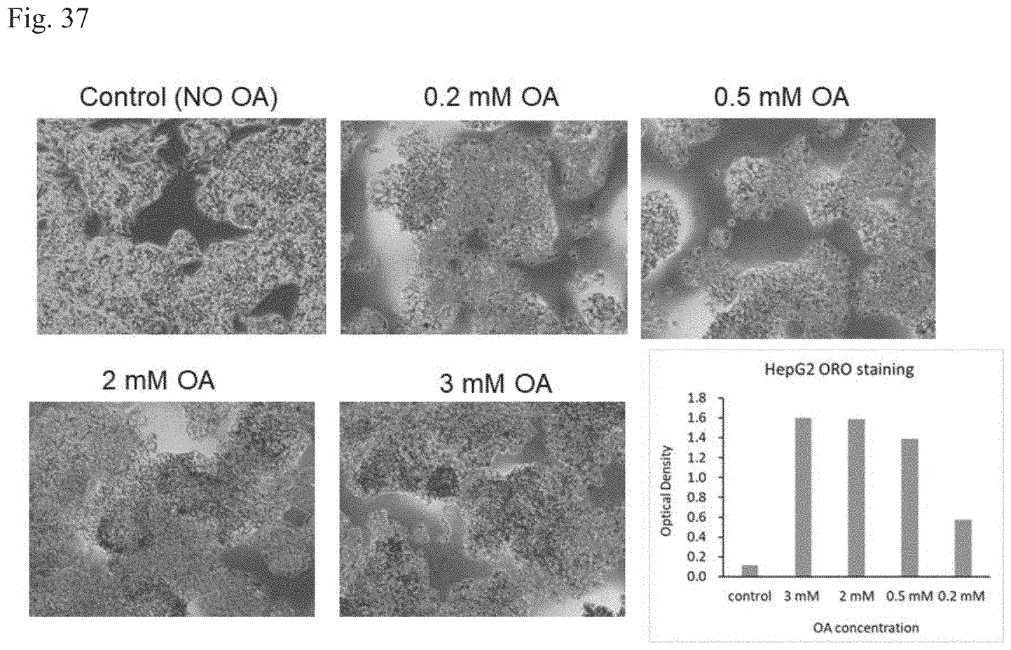

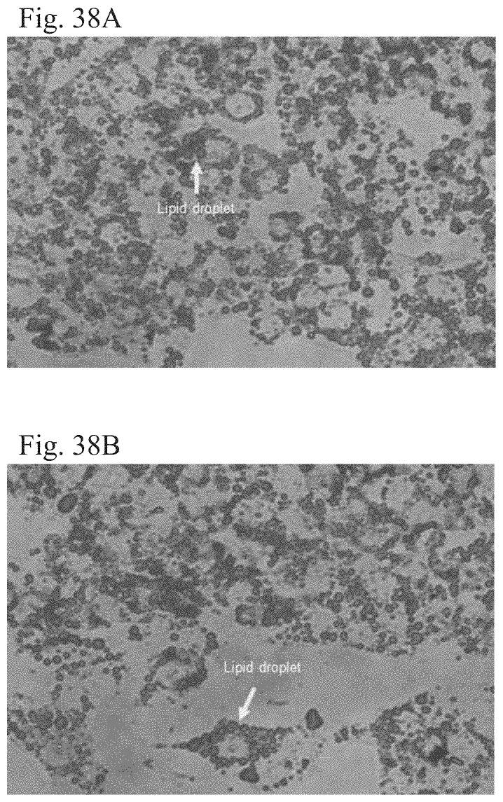

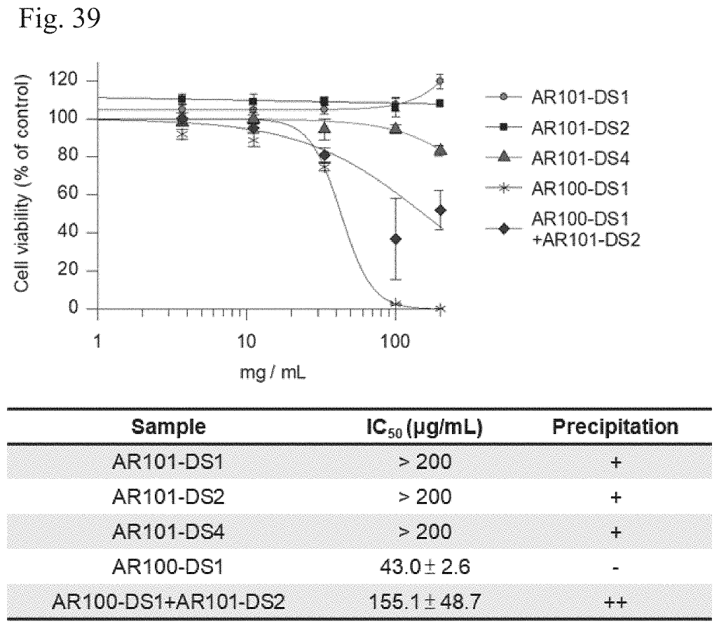

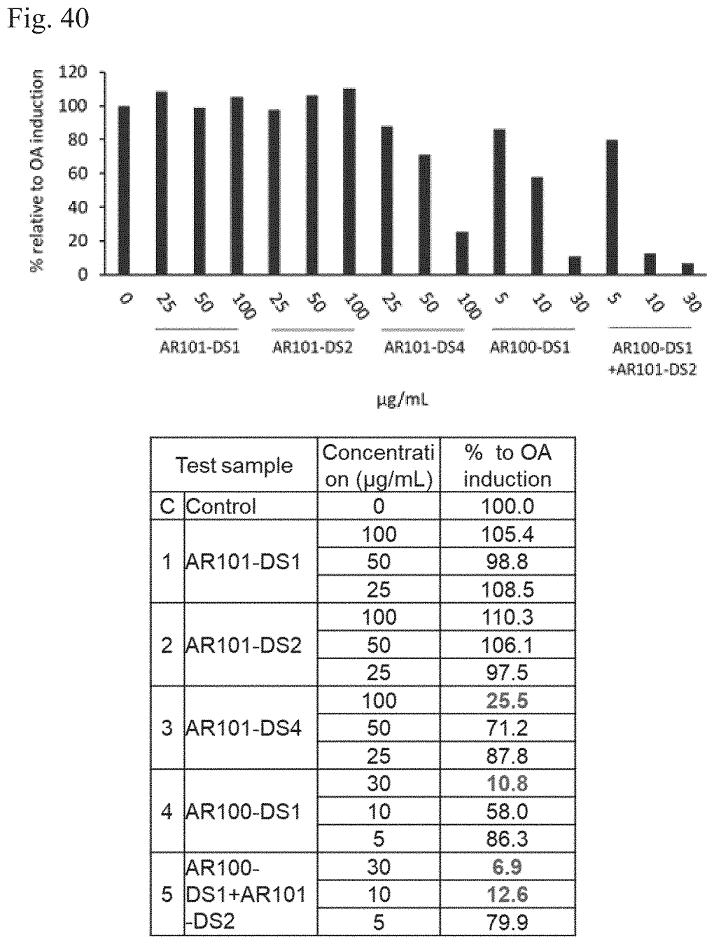

Oleic acid (OA) induced steatosis in HepG2 cells and may consider an in vitro model for human fatty liver disease. HepG2 cells were treated with different concentrations of oleic acid (OA) to induce steatosis. Intracellular lipid was stained by Oil Red O (ORO), and quantified by colorimetric assay. 0.5 mM OA was chosen for further studies (FIGS. 37 and 38). HepG2 cells were treated with test compounds (3.7, 11.1, 33.3, 100 and 200 m/mL) and incubated for 48 h (FIG. 39). HepG2 cells were seeded in 24-well plates and deprived from FBS for 24 h. These cells were then treated with different concentrations of test compounds for 6 h before induced with 0.5 mM OA for 24 h. Cells were gently washed with PBS and fixed using paraformaldehyde at RT, then stained with Oil Red (ORO) solution for 30 min at RT. ORO stain was extracted using pure isopropanol and the optical density was detected at 510 nm (FIG. 40). AR101-DS4, AR100-DS1 and AR100-DS1+AR101-DS2 show inhibition against OA-induced steatosis in HepG2 cells at noncytotoxic concentration.

1. A method of preventing or treating a fibrotic condition, comprising administering an effective amount of composition to a subject in need thereof; wherein the composition comprises 4-fused-rings triterpenes extracted from Antrodia camphorate.

2. The method of claim 1, wherein the 4-fused-rings triterpenes are obtained from an ethanol extract of Antrodia camphorate.

3. The method of claim 1, wherein the 4-fused-rings triterpenes are obtained from an ethyl acetate extract of Antrodia camphorate.

4. The method of claim 1, wherein the 4-fused-rings triterpenes are obtained from a methanol extract of Antrodia camphorate.

5. The method of claim 1, wherein the 4-fused-rings triterpenes are obtained from an organic eluent by introducing a methanol extract of Antrodia camphorate into a normal phase chromatography column, and eluting the column with hexane/ethyl acetate/methanol.

6. The method of claim 5, wherein the organic eluent comprises at least one compound selected from the group consisting following formulae and combinations thereof:

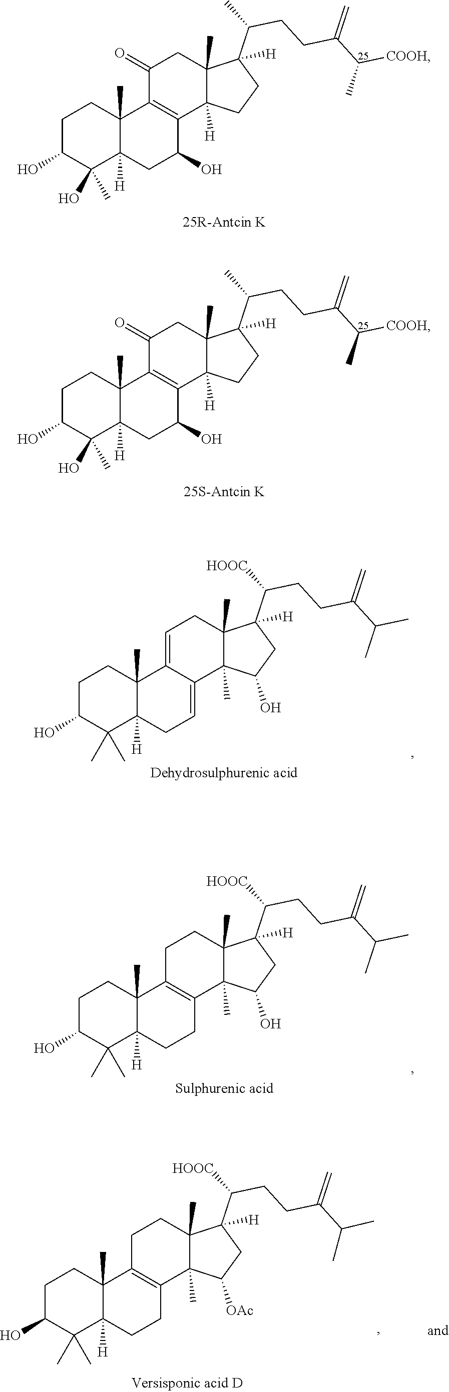

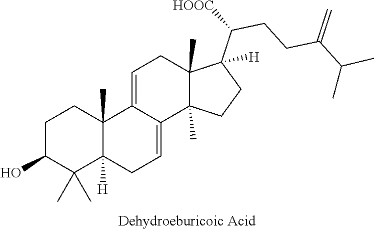 [SMILES: C=C(CC[C@@H](C(=O)O)[C@H]1CC[C@@]2(C)C3=CC[C@H]4C(C)(C)[C@@H](O)CC[C@]4(C)C3=CC[C@]12C)C(C)C]

7. A method of preventing or treating a fibrotic condition, comprising administering to a subject in need thereof an effective amount of a compound selected from the group consisting following formulae and combinations thereof:

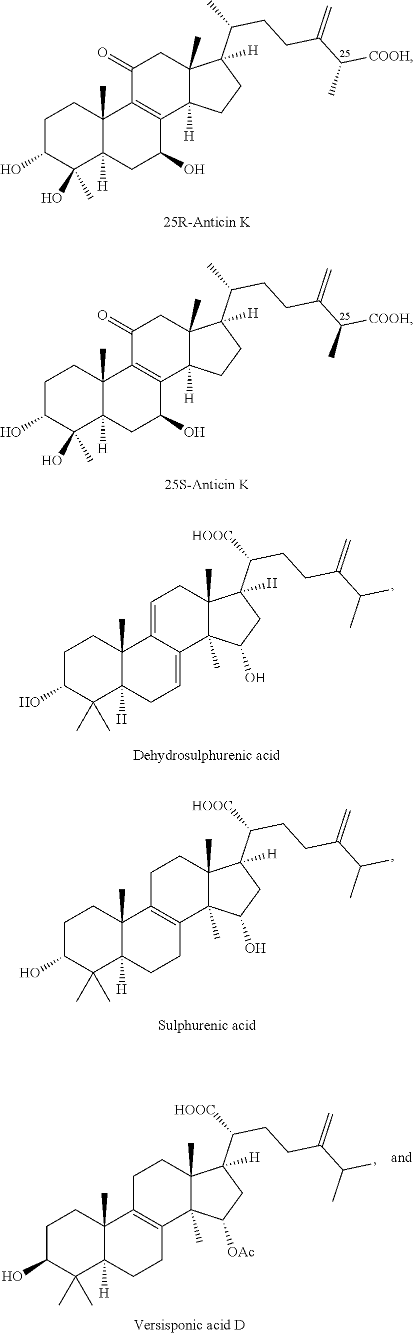

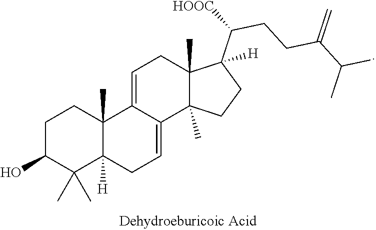

8. The method of claim 1, wherein the fibrotic condition includes hepatic fibrosis, renal fibrosis, vascular fibrosis, pulmonary fibrosis, benign prostatic hyperplasia.

9. The method of claim 1, wherein the composition further alleviates renal dysfunction and renal injury.

10. The method of claim 1, wherein the composition further alleviates non-alcoholic steatohepatitis (NASH), non-alcoholic fatty liver disease (NAFLD), and
inflammation, vacuolation and necrosis in liver.

11. The method of claim 1, wherein the composition in a combination with ovatodiolide further alleviates non-alcoholic steatohepatitis (NASH), non-alcoholic fatty liver disease (NAFLD), and inflammation, vacuolation and necrosis in liver.
